(19) United States
(12) Patent Application Publication
(10) Pub. No.: US 2023/0293666 A1
(43) Pub. Date: Sep. 21, 2023

(54) MANNOSE CONJUGATED CHITOSAN-BASED INFLUENZA NANOVACCINE FORMULATIONS AND USES THEREOF

(71) Applicant: Ohio State Innovation Foundation, Columbus, OH (US)

(72) Inventors: Renukaradhya Gourapura, Columbus, OH (US); Sankar Renu, Columbus, OH (US)

(21) Appl. No.: 18/166,743

(22) Filed: Feb. 9, 2023

Related U.S. Application Data
(60) Provisional application No. 63/308,251, filed on Feb. 9, 2022.

(51) Int. Cl. A61K 39/145; A61K 9/00; A61K 9/51; A61P 31/16; A61K 39/39

(52) U.S. Cl. CPC A61K 39/145; A61K 9/0043; A61K 9/5161; A61K 39/39; A61P 31/16; A61K 2039/5252; C12N 2760/16134

(57) ABSTRACT

Disclosed herein are nanoparticles comprising mannose conjugated chitosan and an inactivated influenza A virus (IAV) antigen, wherein the mannose conjugated chitosan encapsulates the inactivated IAV antigen. In some embodiments, the nanoparticle further comprises tripolyphosphate. Also disclosed are methods of reducing transmission of an influenza A virus, and methods of eliciting an immune response against an influenza A virus, in a subject compared to a control comprising administering to the subject a nanoparticle comprising mannose conjugated chitosan and an inactivated influenza A virus (IAV) antigen, wherein the mannose conjugated chitosan encapsulates the inactivated IAV antigen.

### CROSS-REFERENCE TO RELATED APPLICATIONS

The application claims the benefit of U.S. Provisional Application No. 63/308,251, filed Feb. 9, 2022, which is hereby incorporated herein by reference in its entirety.

### FIELD

The disclosure generally relates to nanoparticles, particularly nanoparticles useable in a vaccine. The nanoparticles are capable of increasing immunity to various influenza A viruses. The disclosure further relates to methods to make and use nanoparticles and vaccines comprising nanoparticles.

### BACKGROUND

Swine influenza is an acute respiratory disease of pigs caused by swine influenza A virus (SwIAV). Pigs are naturally vulnerable to IAV-associated with secondary bacterial infections. Swine IAV is an economic threat to the global pig industry. Commonly circulating SwIAV strains in swine population are H1N1, H1N2, and H3N2. In the United States, periodically human infections are occurred from some of the SwIAVs. In last two decades, triple reassortment SwIAVs have been isolated from pigs, and its-association with human infections have also been documented. The most recent is the 2009 pandemic H1N1 SwIAV spillover to humans. Therefore, vaccination of pigs is a common practice to reduce the influenza burden in swine industry and to avoid the risk of zoonotic transmission to humans. The SwIAV vaccine inoculated into sows protects the herd from infection and heightens the transfer of maternally-derived antibodies (MDA) to offspring through colostrum. However, a number of studies have revealed that MDA offered various levels of protection against IAV infection in piglets. In weaned piglets, MDA interferes with parenteral administered killed/inactivated influenza virus vaccines, resulting in poor induction of antibody responses and documented evidence of vaccine-associated enhanced respiratory disease.

The MDA inhibits the vaccine-induced IgG antibody and does not interfere with the secretory IgA (sIgA) antibody production. Intranasally (IN) administered inactivated IAV vaccine in mice overcomes the MDA interference and provides complete protection in offspring. Influenza viruses use nasal mucosa as a main entry site. Effective vaccines delivered IN trigger the mucosal immunity and offer the frontline defense against the infection. Further, IN vaccination activates the B and T cells in the nasal-associated lymphoid tissues and induce specific antibody and cell-mediated immune responses.

Chitosan is a biocompatible polymer, and its protonated positively charged amino groups electrostatically interact with negative charged mucus sialic acid and epithelial surfaces to become mucoadhesive vehicle. Hence, chitosan nanoparticles (CS NPs) were used as a mucosal vaccine delivery carrier for the poultry and swine vaccines to combat infectious diseases. In protein antigens encapsulated CS NPs, treated immune cells in vitro demonstrate upregulated multiple Toll-like receptors (TLRs), Th1 and Th2 cytokines gene expression. In SwIAV killed antigen loaded CS NPs treated dendritic cells (DCs) observed enhanced secretion of innate, pro-inflammatory and Th1 cytokines, and in IN vaccinated pigs, induction of enhanced cross-reactive mucosal immunity has been observed.

The calcium-dependent (C-type) lectin family mannose receptor (MR) is a carbohydrate binding protein, primarily expressed by the DCs and macrophages. The MR binds to mannosylated protein and the antigens uptaken through MR are efficiently processed and presented through major histocompatibility pathways by DCs. Mannose ligand is internalized by DCs through receptor-mediated endocytosis. In vitro, mannan ligand-coated nanoparticles readily binds to MR expressing cells and internalized. Mannose ligand in mannosylated CS NPs interacts with MR on the surface of macrophages and facilitates its uptake. In vivo, glycosylated nanoparticles rapidly shuttle to the follicular DCs network and are concentrated in germinal centers of lymph nodes thereby triggering the innate immune-mediated recognition pathway and promotes antigen-specific responses. For these reasons, the MR receptor on cells is a possible target for vaccine delivery. What is needed are improved vaccines for use in increasing immunity to various influenza A viruses, particular in pigs.

The compositions and methods disclosed herein address these and other shortcomings in the art.

### SUMMARY

The disclosure herein addresses needs in the art by providing compositions and methods useful in the prevention and treatment of certain infectious agents. The nanoparticles disclosed herein are particularly useful in vaccines administered to pigs to prevent or treat influenza A virus (IAV) infection.

More specifically, mannose conjugated chitosan (mCS) and killed antigen (KAg) encapsulated mCS nanoparticles were targeted to dendritic cells and macrophages which express mannose receptor. In maternal derived antibody (MDA)-positive piglets, prime-boost intranasal inoculation of mCS NPs-KAg vaccine elicited enhanced homologous (H1N2-OH10), heterologous (H1N1-OH7), and heterosubtypic (H3N2-OH4) influenza virus-specific secretory IgA (sIgA) antibody response in nasal passage compared to CS NPs-KAg vaccinates. Upon challenge with a heterologous SwIAV H1N1, the mCS NPs-KAg vaccines augmented H1N2-OH10, H1N1-OH7, and H3N2-OH4 virus-specific sIgA antibody responses in nasal swab, lung lysate, and bronchoalveolar lavage (BAL) fluid; and IgG antibody levels in lung lysate and BAL fluid samples. In the mCS NPs-KAg vaccinates increased H1N2-OH10 but not H1N1-OH7 and H3N2-OH4-specific serum hemagglutination inhibition titers were observed.

Additionally, mCS NPs-KAg vaccine increased specific recall lymphocyte proliferation and cytokines IL-4, IL-10, and IFN_{γ} gene expression compared to CS NPs-KAg and commercial SwIAV vaccinates in tracheobronchial lymph nodes. The mCS NPs-KAg vaccinates cleared the challenge H1N1-OH7 virus load in upper and lower respiratory tract more efficiently when compared to commercial vaccine. The virus clearance was associated with reduced gross lung lesions. Overall, mCS NP-KAg vaccine intranasal immunization in MDA-positive pigs induced a robust cross-reactive immunity and offered protection against influenza virus.

In one aspect, disclosed herein is a nanoparticle comprising mannose conjugated chitosan and an inactivated influenza A virus (IAV) antigen, wherein the mannose conjugated chitosan encapsulates the inactivated IAV antigen. In some embodiments, the nanoparticle further comprises tripolyphosphate. In some embodiments, the nanoparticle reduces nasal shedding of an influenza A virus (IAV). In some embodiments, the nanoparticle increases the amount of IgA antibody in a subject.

In some aspects, disclosed herein is a method of reducing transmission of an influenza A virus (IAV) in a subject comprising administering to the subject a nanoparticle comprising mannose conjugated chitosan and an inactivated influenza A virus (IAV) antigen, wherein the mannose conjugated chitosan encapsulates the inactivated IAV antigen. In some embodiments, the method reduces nasal shedding of the IAV.

In some aspects, disclosed herein is a method of eliciting an immune response against swine influenza A virus in a subject comprising administering to the subject a nanoparticle comprising mannose conjugated chitosan and an inactivated influenza A virus (IAV) antigen, wherein the mannose conjugated chitosan encapsulates the inactivated IAV antigen. In some embodiments, the method elicits an increased amount of IgA antibody in the subject compared to a control.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Additional aspects and advantages of the disclosure will be set forth, in part, in the detailed description and any claims which follow, and in part will be derived from the detailed description or can be learned by practice of the various aspects of the disclosure. It is to be understood that both the foregoing general description and the following detailed description are exemplary and explanatory only and are not restrictive of the disclosure.

The accompanying drawings, which are incorporated in and constitute a part of this specification, illustrate certain examples of the present disclosure and together with the description, serve to explain, without limitation, the principles of the disclosure. Like numbers represent the same element(s) throughout the figures.

FIGS. 1A-1F. Mannose conjugated CS NPs based influenza (mCS NPs-KAg) vaccine augmented cross-reactive secretory IgA while the commercial flu vaccine boosts specific IgG antibodies in MDA-positive pigs. Pigs were vaccinated twice with mCS NPs-KAg or CS NPs-KAg vaccines (containing H1N2-OH10 virus) intranasally or commercial vaccine (containing H1N1, H1N2 and H3N2 viruses) intramuscularly. On the day post vaccination 35 nasal swab and blood samples collected were subjected to antibody analysis. Secretory IgA antibody response in nasal swab against (FIG. 1A) H1N2-OH10; (FIG. 1B) H1N1-OH7; and (FIG. 1C) H3N2-OH4 viruses, and IgG antibody response in serum against (FIG. 1D) H1N2-OH10; (FIG. 1E) H1N1-OH7; and (FIG. 1F) H3N2-OH4 viruses were analyzed by ELISA. Data represent the mean value of three to four pigs ± SEM at all indicated dilutions. Statistical analysis was carried out using two-way ANOVA followed by a Bonferroni test. Each letter on the line graph indicates the significant difference between the groups at the marked sample dilution: b, c and d indicate the difference between mock group compared to commercial vaccine, CS NPs-KAg, and mCS NPs-KAg, respectively; h and i indicate the difference between commercial vaccine compared to CS NPs-KAg and mCS NPs-KAg, respectively. A p < 0.05 was considered statistically significant.

FIGS. 2A-2I. Mannose-conjugated and unconjugated CS NPs based influenza (mCS NPs-KAg and CS NPs-KAg) vaccinates elicited higher cross-reactive secretory IgA antibody response in MDA-positive pigs post challenge infection. Pigs were prime-boost vaccinated with mCS NPs-KAg or CS NPs-KAg vaccine (containing H1N2-OH10 virus) intranasally or commercial vaccine (containing H1N1, H1N2 and H3N2 viruses) intramuscularly and at day post vaccination 35 challenged with a heterologous H1N1-OH7 virus. On day six post-challenge secretory IgA antibodies in nasal swab, lung lysate, and BAL fluid samples against (FIG. 2A, FIG. 2D, FIG. 2G) H1N2-OH10; (FIG. 2B, FIG. 2E, FIG. 2H) H1N1-OH7; and (FIG. 2C, FIG. 2F, FIG. 2I) H3N2-OH4 viruses were analyzed by ELISA. Data represent the mean value of three to four pigs ± SEM at all indicated dilutions. Statistical analysis was carried out using two-way ANOVA followed by a Bonferroni test. Each letter on the line graph indicates the significant difference between the groups at the marked sample dilution: b, c and d indicate the difference between mock group compared to commercial vaccine + Ch, CS NPs-KAg + Ch, and mCS NPs-KAg + Ch, respectively; e, f and g indicate the difference between mock + Ch group compared to commercial vaccine + Ch, CS NPs-KAg + Ch and mCS NPs-KAg + Ch, respectively; h and i indicate the difference between commercial vaccine + Ch compared to CS NPs-KAg + Ch and mCS NPs-KAg + Ch, respectively; j indicates the difference between CS NPs-KAg + Ch compared to mCS NPs-KAg + Ch. A p < 0.05 was considered statistically significant. Ch- Challenge.

FIGS. 3A-3I. Commercial swine flu vaccine increased the cross-reactive serum IgG, and mannose-conjugated and unconjugated CS NPs influenza (mCS NPs-KAg and CS NPs-KAg) vaccines augmented IgG antibody response in lung lysate and BAL fluid of MDA-positive pigs. Pigs were vaccinated with mCS NPs-KAg or CS NPs-KAg vaccine (containing H1N2-OH10 virus) intranasally or commercial vaccine (containing H1N1, H1N2 and H3N2 viruses) intramuscularly and at day post vaccination 35 challenged with a H1N1-OH7 virus. On day six post challenge IgG antibody response in serum, lung lysate and BAL fluid samples against (FIG. 3A, FIG. 3D, FIG. 3G) H1N2-OH10; (FIG. 3B, FIG. 3E, FIG. 3H) H1N1-OH7; and (FIG. 3C, FIG. 3F, FIG. 3I) H3N2-OH4 viruses were analyzed by ELISA. Data represent the mean value of three to four pigs ± SEM at all indicated dilutions. Statistical analysis was carried out using two-way ANOVA followed by a Bonferroni test. Each letter on the line graph indicates the significant difference between the groups at the marked sample dilution: b, c and d indicate the difference between mock group compared to commercial vaccine + Ch, CS NPs-KAg + Ch, and mCS NPs-KAg + Ch, respectively; e, f and g indicate the difference between mock + Ch group compared to commercial vaccine + Ch, CS NPs-KAg + Ch, and mCS NPs-KAg + Ch, respectively; h and i indicate the difference between commercial vaccine + Ch compared to CS NPs-KAg + Ch and mCS NPs-KAg + Ch, respectively; j indicates the difference between CS NPs-KAg + Ch compared to mCS NPs-KAg + Ch. A p < 0.05 was considered statistically significant. Ch- Challenge. FIGS. 4A-4C. Hemagglutination inhibition (HI) antibody titers in MDA-positive pigs vaccinated with mannose-conjugated and unconjugated CS NPs based influenza (mCS NPs-KAg and CS NPs-KAg) or commercial flu vaccine and challenged at day post vaccination 35 with the H1N1-OH7 virus. On day six post challenge HI antibody titer in serum against (FIG. 4A) H1N2-OH10; (FIG. 4B) H1N1-OH7; and (FIG. 4C) H3N2-OH4 viruses were measured. Data represent the mean value of three to four pigs ± SEM. Statistical analysis was carried out using one-way ANOVA followed by Tukey’s post hoc comparison test. Asterisk refers to statistical difference between the two indicated groups (* p < 0.05, ** p < 0.01, and *** p < 0.001). Ch- Challenge.

FIGS. 5A-5D. Augmented cell-mediated immune response in MDA-positive pigs vaccinated with mannose-conjugated and unconjugated CS NPs based influenza (mCS NPs-KAg and CS NPs-KAg) vaccines and not commercial flu vaccine. At day six post challenge infection with H1N1-OH7 virus the isolated MNCs of tracheobronchial lymph nodes (TBLN) were stimulated with vaccine virus (H1N2-OH10). (FIG. 5A) Lymphocytes proliferation stimulation index in TBLN was analyzed by ELISA. TBLN tissues stored in RNAlater were analyzed by qRT-PCR for the expression of mRNA of (FIG. 5B) IL-10; (FIG. 5C) IL-4; and (FIG. 5D) IFNγ. Data represent the mean value of three to four pigs ± SEM. Statistical analysis was carried out using one-way ANOVA followed by Tukey’s post hoc comparison test. Asterisk refers to statistical difference between the two indicated groups (* p < 0.05). Ch- Challenge.

FIGS. 6A-6F. Mannose-conjugated and unconjugated CS NPs based influenza (mCS NPs-KAg and CS NPs-KAg) vaccines reduced/cleared the heterologous challenge H1N1 virus in MDA-positive pigs. Pigs were prime-boost vaccinated with mCS NPs-KAg and CS NPs-KAg vaccines (containing H1N2-OH10 virus) intranasally or commercial vaccine (containing H1N1, H1N2 and H3N2 viruses) intramuscularly and at day 35 post vaccination challenged with a heterologous H1N1-OH7 virus. At day post challenge (DPC)-4 and -6 the live H1N1-OH7 virus titers were analyzed in (FIG. 6A and FIG. 6B) Nasal swab; (FIG. 6C) BAL fluid (DPC-6); and (FIG. 6D) Lung lysate (DPC-6). (FIG. 6E) Representative ventral lung picture of experimental pigs showing the dark brown sites of consolidation indicated by arrows. (FIG. 6F) Macroscopic lung lesions score. Data represent the mean value of three to four pigs ± SEM. Statistical analysis was carried out using one-way ANOVA followed by Tukey’s post hoc comparison test. Asterisk refers to statistical difference between the two indicated groups (* p < 0.05, and ** p < 0.01). Ch-Challenge.

FIGS. 7A-7H. Augmented cell-mediated immune response in MDA-positive pigs vaccinated with mannose-conjugated and unconjugated CS NPs based influenza (mCS NPs-KAg and CS NPs-KAg) vaccines and not commercial flu vaccine. At day six post challenge infection with H1N1-OH7 virus the isolated MNCs of tracheobronchial lymph nodes (TBLN) were stimulated with vaccine (H1N2-OH10) virus; peripheral blood mononuclear cells (PBMCs) isolated at day 35 post vaccination (pre-challenge) were stimulated with vaccine (H1N2-OH10) or challenge (H1N1-OH7) virus for 72 h. (FIG. 7A) Lymphocytes proliferation stimulation index in TBLN was analyzed by ELISA. Flow cytometry analysis was performed to determine the frequency of different lymphocyte subsets in TBLN: (FIG. 7B) Cytotoxic T lymphocytes (CTLs); IFNγ secreting (FIG. 7C) CTLs and (FIG. 7D) T-helper/memory cells. In PBMCs stimulated with H1N1-OH7 virus the frequency of (FIG. 7E) Late effector CTLs (CD3⁺CD4⁻ CD8α⁺β⁺Perforin⁺CD27⁻) and (FIG. 7F) Effector memory cells (CD3⁺CD4⁺CD8α⁺β⁻CD27⁻). In PBMCs stimulated with H1N2-OH10 virus the frequency of (FIG. 7G) IFNγ secreting CTLs and (FIG. 7H) Effector memory cells (CD3⁺CD4⁺CD8α⁺β⁻CD27⁻). Data represent the mean value of three to four pigs ± SEM. Statistical analysis was carried out using one-way ANOVA followed by Tukey’s post hoc comparison test. Asterisk refers to statistical difference between the two indicated groups (* p < 0.05, and ** p < 0.01). Ch- Challenge.

FIG. 8. Experimental pig groups had the influenza virus specific maternal IgG antibody in the serum. Pregnant sows were vaccinated with commercial influenza vaccine at 2 and 5 weeks before farrowing. Serum collected from weaned piglets at three weeks of age was serial tenfold diluted and analyzed for the H1N2-OH10 virus specific IgG antibody level. Data represent the mean value of three to four pigs ± SEM at all indicated dilutions. Statistical analysis was carried out using one-way ANOVA followed by Tukey’s post hoc comparison test.

### DETAILED DESCRIPTION

The disclosure herein addresses needs in the art by providing compositions and methods useful in the prevention and treatment of certain infectious agents. The nanoparticles disclosed herein are particularly useful in vaccines administered to pigs to prevent or treat influenza A virus (IAV) infection.

Those skilled in the relevant art will recognize and appreciate that many changes can be made to the various embodiments of the invention described herein, while still obtaining the beneficial results of the present disclosure. It will also be apparent that some of the desired benefits of the present disclosure can be obtained by selecting some of the features of the present disclosure without utilizing other features. Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

### Terminology

Unless defined otherwise, all technical and scientific terms used herein have the same meaning as commonly understood to one of ordinary skill in the art to which this disclosure belongs. The term “comprising” and variations thereof as used herein is used synonymously with the terms “including,” “containing,” and variations thereof and are open, non-limiting terms. Although the terms “comprising,” “including,” and “containing” have been used herein to describe various embodiments, the terms “consisting essentially of’ and “consisting of” can be used in place of “comprising,” “including,” and “containing” to provide for more specific embodiments and are also disclosed.

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular nanoparticle is disclosed and discussed and a number of modifications that can be made to the nanoparticle are discussed, specifically contemplated is each and every combination and permutation of the nanoparticle and the modifications that are possible unless specifically indicated to the contrary. Thus, if a class of nanoparticles A, B, and C are disclosed as well as a class of nanoparticles D, E, and F and an example of a combination nanoparticle, or, for example, a combination nanoparticle comprising A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

It is understood that the compositions disclosed herein have certain functions. Disclosed herein are certain structural requirements for performing the disclosed functions, and it is understood that there are a variety of structures which can perform the same function which are related to the disclosed structures, and that these structures will ultimately achieve the same result.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

As used in the specification and claims, the singular form “a,” “an,” and “the” include plural references unless the context clearly dictates otherwise. For example, the term “an agent” includes a plurality of agents, including mixtures thereof.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

An “immunogenic composition” is a composition of matter suitable for administration to a human or animal subject that is capable of eliciting a specific immune response, e.g., against a pathogen, such as swine influenza A virus. As such, an immunogenic composition includes one or more antigens (for example, whole purified virus or antigenic subunits, e.g., polypeptides, thereof) or antigenic epitopes. An immunogenic composition can also include one or more additional components capable of eliciting or enhancing an immune response, such as an excipient, carrier, and/or adjuvant. In certain instances, immunogenic compositions are administered to elicit an immune response that protects the subject against symptoms or conditions induced by a pathogen. In some cases, symptoms or disease caused by a pathogen is prevented (or treated, e.g., reduced or ameliorated) by inhibiting replication of the pathogen following exposure of the subject to the pathogen. In the context of this disclosure, the term immunogenic composition will be understood to encompass compositions that are intended for administration to a subject or population of subjects for the purpose of eliciting a protective or palliative immune response against the virus (that is, vaccine compositions or vaccines).

An “antigen” is a compound, composition, or substance that can stimulate the production of antibodies and/or a T cell response in an animal, including compositions that are injected, absorbed or otherwise introduced into an animal. The term “antigen” includes all related antigenic epitopes. The term “epitope” or “antigenic determinant” refers to a site on an antigen to which B and/or T cells respond. The “dominant antigenic epitopes” or “dominant epitope” are those epitopes to which a functionally significant host immune response, e.g., an antibody response or a T-cell response, is made. Thus, with respect to a protective immune response against a pathogen, the dominant antigenic epitopes are those antigenic moieties that when recognized by the host immune system result in protection from disease caused by the pathogen. The term “T-cell epitope” refers to an epitope that when bound to an appropriate MHC molecule is specifically bound by a T cell (via a T cell receptor). A “B-cell epitope” is an epitope that is specifically bound by an antibody (or B cell receptor molecule). An antigen can also affect the innate immune response.

An “immune response” is a response of a cell of the immune system, such as, but not limited to, a B cell, T cell, or monocyte, to a stimulus. An immune response can be a B cell response, which results in the production of specific antibodies, such as antigen specific neutralizing antibodies. An immune response can also be a T cell response, such as a CD4+ T cell response or a CD8+ T cell response. In some cases, the response is specific for a particular antigen (that is, an “antigen-specific response”). An immune response can also include the innate response. If the antigen is derived from a pathogen, the antigen-specific response is a “pathogen-specific response.” A “protective immune response” is an immune response that inhibits a detrimental function or activity of a pathogen, reduces infection by a pathogen, or decreases symptoms (including death) that result from infection by the pathogen. A protective immune response can be measured, for example, by the inhibition of viral replication or plaque formation in a plaque reduction assay or ELISA-neutralization assay, or by measuring resistance to pathogen challenge in vivo.

The abbreviation “KAg” stands for killed antigen and represents the killed or inactivated virus or portions of a virus. The inactivated antigen comprises one or more immunogenic viral proteins and therefore the inactivated antigen can be an inactivated whole virus or a portion of a virus that is inactivated.

The abbreviation “CNP-KAg” or “CNPs-KAg” stands for chitosan nanoparticle-killed antigen. This represents the chitosan nanoparticle having encapsulated inactivated influenza A virus antigen.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The term “prevent” refers to a treatment that forestalls or slows the onset of a disease or condition or reduced the severity of the disease or condition.

“Administration” to a subject includes any route of introducing or delivering to a subject an agent. Administration can be carried out by any suitable route, including oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation, via an implanted reservoir, parenteral (e.g., subcutaneous, intravenous, intramuscular, intra-articular, intra-synovial, intrasternal, intrathecal, intraperitoneal, intrahepatic, intralesional, and intracranial injections or infusion techniques), and the like. “Concurrent administration”, “administration in combination”, “simultaneous administration” or “administered simultaneously” as used herein, means that the compounds are administered at the same point in time or essentially immediately following one another. In the latter case, the two compounds are administered at times sufficiently close that the results observed are indistinguishable from those achieved when the compounds are administered at the same point in time. “Systemic administration” refers to the introducing or delivering to a subject an agent via a route which introduces or delivers the agent to extensive areas of the subject’s body (e.g. greater than 50% of the body), for example through entrance into the circulatory or lymph systems. By contrast, “local administration” refers to the introducing or delivery to a subject an agent via a route which introduces or delivers the agent to the area or area immediately adjacent to the point of administration and does not introduce the agent systemically in a therapeutically significant amount. For example, locally administered agents are easily detectable in the local vicinity of the point of administration but are undetectable or detectable at negligible amounts in distal parts of the subject’s body. Administration includes self-administration and the administration by another.

“Pharmaceutically acceptable” component can refer to a component that is not biologically or otherwise undesirable, e.g., the component may be incorporated into a pharmaceutical formulation of the invention and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed.

### Mannose Conjugated Chitosan Nanoparticle Compositions

It is understood that the nanoparticles of the present disclosure can be used in combination with the various compositions, methods, products, kits, and applications disclosed herein.

Disclosed herein is a novel nanoparticle. The nanoparticle comprises mannose conjugated chitosan, and further comprises an inactivated influenza A virus (IAV) antigen. The nanoparticle is configured such that the mannose conjugated chitosan encapsulates the inactivated IAV antigen.

The nanoparticles disclosed herein have numerous advantageous properties, including but not limited to, capacity to stimulate desirable immune responses including increased secretion of mucosal IgA antibodies, to reduce viral transmission (e.g., via nasal shedding route), and to provide effective immunity to one or more homologous, heterologous, and heterosubtypic influenza A viruses (IAVs). The nanoparticles further are comprised of a biocompatible, biodegradable, mucoadhesive, polycationic, and immunomodulatory design, can deliver highly immunostimulatory antigens, and have desirable size and encapsulation efficiency.

The nanoparticle comprises mannose conjugated chitosan. Chitosan is a polysaccharide containing linkages of acetylated and deacetylated glucosamine and is commercially available from numerous sources. Chitosan is derived from chitin, an N-acetylglucosamine polymer common in fungi, arthropods, and other organisms. Chitosan is often formed from alkaline treatment of chitin in crustacean shells. Chitosan is biocompatible, non-toxic, and biodegradable, and is thus advantageous for use as a polymer to form a nanoparticle for administration to a mammalian subject.

Chitosan is typically composed of β-(1-4)-linked d-glucosamine and N-acetyl-d-glucosamine randomly distributed within the polymer. The degree of deacetylation of glucosamine subunits can affect the properties of chitosan, e.g., immunogenic properties. The degree of deacetylation can be described by the molar fraction of deacetylated units or percentage of deacetylation, and the molecular weight of chitosan. See Cheung et. al., Mar Drugs. 2015 Aug; 13(8): 5156-5186. In some embodiments, the nanoparticles comprise chitosan that is at least 50% deacetylated. In some embodiments, the nanoparticles comprise chitosan that is at least 55%, at least 60%, at least 65%, at least 70%, at least 75%, at least 80%, at least 85%, at least 90%, at least 95%, at least 98%, or at least 99% deacetylated. In some embodiments, the chitosan has a molecular weight up to 500.000 Da, up to 375.000 Da, up to 300.000 Da, up to 250.000 Da, up to 220.000 Da, or up to 190.000 Da. In some embodiments, the chitosan has a molecular weight of at least 10.000 Da, at least 20.000 Da, at least 30.000 Da, at least 40.000 Da, or at least 50.000 Da. In some embodiments, the chitosan has a molecular weight ranging from about 1.000 Da to about 500.000 Da, from about 10.000 Da to about 375.000 Da, from about 20.000 Da to about 300.000 Da, from about 30.000 Da to about 250.000 Da, from about 40.000 Da to about 220.000 Da, or from about 50.000 Da to about 190.000 Da.

Chitosan can be used in combination with additional compounds and polymers suitable for forming a nanoparticle. For example, the chitosan can be combined with natural or synthetic polymers, lipids, saccharides and polysaccharides, proteins, ligands such as targeting molecules, and other molecules or polymers suitable for forming a nanoparticle within the spirit of the invention.

Mannose (or mannose ligand) is a carbohydrate with a molecular formula C₆H₁₂C₆ and is also known under its IUPAC nomenclature as (2S,3S,4R,5R)-Pentahydroxyhexanal, (2R,3R,4S,5S)-Pentahydroxyhexanal. Mannose occurs in two diastereomeric isoforms, D-Mannose and L-Mannose (CAS numbers 3458-28-4 for D-mannose and 10030-80-5 for L-mannose).

In some embodiments, the nanoparticle further comprises a crosslinking agent. The crosslinking agent can crosslink chitosan polymers to aid in formation of the nanoparticle. In some embodiments, the crosslinking agent is negatively charged. In some embodiments, the crosslinking agent comprises tartaric acid (e.g., L(+)-tartaric acid), a carboxy-modified polyethylene glycol (e.g., poly(ethylene glycol) bis(carboxymethyl) ether), an acetic acid derivative (e.g., 4-phenylenediacetic acid), an isophthalic acid derivative (e.g., 5- sulfoisophthalic acid), or tripolyphosphate (TPP).

In some embodiments, the crosslinking agent comprises TPP. Because TPP has low toxicity in vivo, the useable amounts of TPP are not particularly limited. The nanoparticle can comprise a chitosan:TPP ratio, on a weight per volume basis, up to about 100:1, up to about 50:1, up to about 25:1, up to about 10:1, up to about 5:1, up to about 4:1, up to about 3:1, up to about 2:1, up to about 1:1, up to about 0.9:1, up to about 0.8:1, up to about 0.7:1, up to about 0.6:1, up to about 0.5:1, up to about 0.1:1, up to about 0.05:1, or up to about 0.01:1. As a non-limiting example, the nanoparticle can comprise about 1% chitosan (w/v) and about 0.5% TPP (w/v), resulting in a chitosan:TPP ratio, on a weight per volume basis, of up to about 2:1.

In some embodiments, the empty or unloaded nanoparticle (nanoparticle which does not contain encapsulated inactivated IAV antigen) comprises at least 50% by weight chitosan. In some embodiments, the unloaded nanoparticle comprises at least 55%, at least 60%, at least 65%, at least 70%, at least 75%, at least 80%, at least 85%, at least 90%, at least 95%, or at least 99% chitosan. Percent chitosan can be determined by several measures, including percent by dry weight, percent by weight per volume, percent by molar ratio or molarity, or by other known measures.

The nanoparticle comprises an inactivated influenza A virus (IAV) antigen. Any suitably immunogenic inactivated swine influenza A virus or swine influenza A virus antigen can be used. For example, the swine influenza A virus antigen can be a swine influenza A virus surface glycoprotein. Examples of immunogenic antigens include recombinantly derived hemagglutinin, neuraminidase, nucleocapsid and matrix proteins. The swine influenza A virus antigen can be recombinantly derived. In some embodiments, a portion of the inactivated IAV antigen is derived from an IAV which is not a swine IAV (e.g., an avian IAV or human IAV). As non-limiting examples, the inactivated IAV antigen can comprise a swine hemagglutinin protein and an avian neuraminidase protein, or alternatively can comprise an inactivated whole swine IAV and an inactivated whole human IAV. The inactivated IAV antigen can be the entire inactivated virus or can be a portion of the inactivated virus (e.g., all or portions of hemagglutinin and/or neuraminidase proteins). In some embodiments, the inactivated IAV antigen is from an H1N1, H1N2 or H3N2 IAV; however, any suitable IAV can be used for preparation of the inactivated IAV antigen.

In some embodiments, the inactivated influenza A virus (IAV) antigen is an inactivated whole influenza A virus. In some embodiments, the inactivated influenza A virus (IAV) antigen is an inactivated whole swine influenza A virus.

As used herein, an “inactivated” virus is a virus grown in culture which, by any number of suitable methods, is caused to lose the ability to replicate. Thus, an inactivated virus cannot replicate in vivo or in vitro. The term is intended to encompass a virus which is killed. In one embodiment, the swine influenza A virus is inactivated by UV light. Other means of inactivation include chemical, heat, or radioactivity.

The nanoparticles are typically mucoadhesive, meaning the nanoparticles have the capability to adhere to mucus or mucosal surfaces in a subject. Cationic chitosan is a mucoadhesive polymer due to its positive charges. Mucoadhesion is advantageous for mucosal drug delivery because it can prolong the residence time of the nanoparticles and increase the half-time of mucociliary clearance at epithelial surfaces.

In some embodiments, the nanoparticle has a positive zeta potential. In some embodiments, the nanoparticle has a net positive charge at neutral pH. In some embodiments, the nanoparticle comprises one or more amine moieties at its surface. In some embodiments, the nanoparticle comprises polymerized, positively charged glucosamine monomers at its surface.

The nanoparticle is configured such that the mannose conjugated chitosan encapsulates the inactivated IAV antigen. By “encapsulated,” it is meant there is a physical encasing of the inactivated IAV antigen in the nanoparticle, such that access to the inactivated IAV antigen is generally blocked. In some embodiments, the nanoparticle comprising mannose conjugated chitosan encapsulates the inactivated IAV antigen such that the composition can elicit an immune response. However, due to inefficiencies in the encapsulation process, some inactivated IAV antigen can be associated on the outer surface of the nanoparticle.

In some embodiments, the nanoparticle is formed by self-assembly. Self-assembly refers to the process of forming a nanoparticle using components that will orient themselves in a predictable manner, thereby forming nanoparticles predictably and reproducibly. In some embodiments, the nanoparticles are formed using amphiphilic biomaterials which orient themselves with respect to one another to form nanoparticles of predictable dimension, constituents, and placement of constituents. In some embodiments, the nanoparticle is formed by ionic gelation.

Nanoparticles can aid the delivery of the inactivated IAV antigen and/or can also be immunogenic. Delivery can be to a particular site of interest, e.g., the mucosa. In some embodiments, the nanoparticle can create a timed release of the inactivated IAV antigen to enhance and/or extend the immune response.

The nanoparticle can have a diameter within the nanometer range (e.g., from 1 nm to 1.000 nm). However, the diameter of the nanoparticle is not specifically limited to the nanometer range and, in some embodiments, can be larger than 1.000 nm (e.g., up to 1.500 nm, up to 2.000 nm, or up to 3.000 nm or more). In some embodiments, the nanoparticle has a diameter of 1.000 nm or less, 750 nm or less, 500 nm or less, 400 nm or less, 300 nm or less, or 200 nm or less. In some embodiments, the nanoparticle has a diameter from 10 nm to less than 1.000 nm. Optionally, the nanoparticle has a diameter from 100 nm to 900 nm, from 200 nm to 800 nm, from 300 nm to 700 nm, from 400 nm to 600 nm, or from 450 nm to 550 nm. In some, embodiments, the nanoparticle has a diameter of about 500 nm. As discussed herein, the diameter can refer to an average diameter of a plurality of nanoparticles.

The encapsulation efficiency of the nanoparticle for inactivated IAV antigen can vary. Encapsulation efficiencies are generally calculable based on the amount of inactivated IAV antigen used in the encapsulation procedure and the amount of inactivated IAV antigen which remains in solution after the nanoparticles are removed from the encapsulation solution. The nanoparticle can have an encapsulation efficiency of 60% or greater of the inactivated IAV antigen. In some embodiments, the nanoparticle has an encapsulation efficiency of 65% or greater, 70% or greater, or 75% or greater of the inactivated IAV antigen. In some embodiments, the nanoparticle has an encapsulation efficiency of about 67% of the inactivated IAV antigen.

In some embodiments, the nanoparticle composition can include unencapsulated inactivated IAV antigen. For instance, the nanoparticle can in some embodiments further comprise inactivated IAV antigen associated on the outer surface of the nanoparticle. The nanoparticles can be administered as nanoparticles which comprise only encapsulated inactivated IAV antigen (e.g., further purified nanoparticles), as nanoparticles comprising encapsulated inactivated IAV antigen and unencapsulated but associated (e.g., nanoparticle outer surface-associated) inactivated IAV antigens, or mixtures thereof.

The nanoparticle can have properties expressed in comparison to a control. As used herein, a “control” refers to a composition which can be administered to a subject to provide comparative data. Alternatively, a control can be a collection of values used as a standard applied to one or more subjects (e.g., a general number or average that is known and not identified in the method using a sample). In some embodiments, the control comprises an empty nanoparticle (e.g., a nanoparticle comprising chitosan, but which contains no encapsulated or associated inactivated IAV antigen). In some embodiments, the control comprises a solution, culture medium, or carrier in which nanoparticles can be added (e.g., water, DMEM). In some embodiments, the control comprises unencapsulated inactivated IAV antigen (KAg), typically in combination with a solution, culture medium, or carrier. In some embodiments, the control can be a different nanoparticle comprising an inactivated IAV antigen (e.g., a PLGA-containing nanoparticle) or a vaccine comprising a nanoparticle comprising an inactivated IAV antigen.

The nanoparticle can be used to prevent or treat influenza A virus infections in a subject. Typically, influenza A virus infection (or exposure thereto) occurs subsequent to administering the nanoparticle to a subject. The nanoparticle can comprise an inactivated IAV antigen which is homologous, heterologous, or heterosubtypic to the influenza A virus causing infection. As used herein, an influenza A virus is “homologous” to the inactivated IAV antigen if the influenza A virus contains the same hemagglutinin (H) and neuraminidase (N) surface proteins as the H and N surface proteins of the virus from which the inactivated IAV antigen is derived (e.g., an inactivated IAV antigen from an H1N2 influenza A virus is homologous to an H1N2 influenza A virus). As used herein, an influenza A virus is “heterologous” to the inactivated IAV antigen if the influenza A virus contains the same hemagglutinin (H) surface protein but a different neuraminidase (N) surface protein as compared to the H and N surface proteins of the virus from which the inactivated IAV antigen is derived (e.g., an inactivated IAV antigen from an H1N2 influenza A virus is heterologous to an H1N1 influenza A virus). As used herein, an influenza A virus is “heterosubtypic” to the inactivated IAV antigen if the influenza A virus contains a different hemagglutinin (H) surface protein as compared to the H surface protein of the virus from which the inactivated IAV antigen is derived (e.g., an inactivated IAV antigen from an H1N2 influenza A virus is heterosubtypic to an H3N2 influenza A virus). As such, a heterosubtypic relationship can include the same or different neuraminidase (N) surface protein.

In some embodiments, the nanoparticle prevents or reduces the symptoms of infection by more than one IAV, each of which are separately homologous, heterologous, or heterosubtypic to the inactivated IAV antigen comprised in the nanoparticle. In some embodiments, the IAV causing infection comprises H1N1, H1N2, H2N2, H2N3, H3N1, H3N2, H3N8, H5N1, H5N2, H5N3, H5N6, H5N8, H5N9, H6N1, H7N1, H7N2, H7N3, H7N4, H7N7, H7N9, H9N2, or H10N7 IAV. In some embodiments, the IAV is a swine IAV. In some embodiments, the IAV comprises H1N1, H1N2, H3N1, H3N2, H2N3, H4N6, or H9N2 IAV. In some embodiments, the IAV comprises an H1N1, H1N2, or H3N2 IAV. However, in some embodiments, the nanoparticles elicit cross-reactive responses to a range of IAVs (e.g., more than one) and thus, the IAV, or combinations thereof, need not be particularly limited.

The nanoparticle can reduce transmission of an IAV. As used herein, an IAV (e.g., an IAV which is reduced in nasal shedding, or for which immune responses are generated against) is an infectious IAV, particularly a swine IAV. An infectious IAV has the capability of infecting a subject, particularly a pig, and to cause symptoms of infection therein. In some embodiments, the nanoparticle reduces nasal shedding of an IAV compared to a control. Viral nasal shedding is an important mode of transmission of IAVs. Reduced nasal shedding can be determined in a sample collected from the upper respiratory tract of a subject by any suitable method, for example by nasal swab of shedding mucous.

The nanoparticles can reduce the amount of IAV in the upper respiratory tract of a subject by an amount quantifiable by any suitable method. In some embodiments, the amount of reduction of IAV in the upper respiratory tract of a subject is quantifiable by the tissue culture infective dose (TCID₅₀/mL) method. See, e.g., Reed et al., Am. J. Hyg., 27(3):493-7 (1938). In some embodiments, the nanoparticle reduces the amount of the IAV in an upper respiratory tract by at least 1×10¹ TCID₅₀/mL compared to a control. In some embodiments, the nanoparticle reduces the amount of the IAV in an upper respiratory tract, as compared to a control, by at least 1×10^{1.2}, at least 1×10^{1.4}, at least 1×10^{1.5}, at least 1×10^{1.6}, at least 1×10^{1.8}, at least 1×10^{2.0}, at least 1×10^{2.5}, at least 1×10^{3.0}, at least 1×10^{3.5}, at least 1×10^{4.0}, at least 1×10^{4.5}, or at least 1×10^{5.0} TCID₅₀/mL, or more.

The reduction in the amount of the IAV in the upper respiratory tract of a subject administered with the nanoparticles can occur shortly after exposure to, or infection with, the IAV. In some embodiments, the amount of IAV in the upper respiratory tract of a subject can be reduced within one, two, three, four, five, six, or seven days of exposure to the IAV. In a particular embodiment, the nanoparticle reduces the amount of IAV in the upper respiratory tract of a subject within four days of exposure to the IAV. In some or further embodiments, the nanoparticle further reduces the amount of IAV in the upper respiratory tract within six days of exposure to the IAV. In some embodiments, the IAV in a subject administered with the nanoparticles is reduced to below clinical detection levels at least one, two, three, four, five, six, seven, or more days earlier than in a subject administered with a control. In some embodiments, the nanoparticle prevents an increase in the amount of infecting IAV to a level which causes symptoms of the infection (e.g., flu-like symptoms).

In some embodiments, the nanoparticle reduces the amount of IAV in the lower respiratory tract, compared to a control. A sample can be collected from the lower respiratory tract of a subject by any suitable method, for example by bronchoalveolar lavage (BAL) of the lungs to obtain BAL fluid. In some embodiments, the reduced amount of an IAV in the lower respiratory tract can be in addition to reduced nasal shedding of the IAV.

The amount of reduction of IAV in the lower respiratory tract of a subject administered with the nanoparticles can be quantified by any suitable method (e.g., by TCID₅₀/mL). In some embodiments, the nanoparticle reduces the amount of the IAV in the lower respiratory tract (e.g., BAL fluid) of a subject by at least 1×10¹ TCID₅₀/mL compared to a control. In some embodiments, the nanoparticle reduces the amount of the IAV in the lower respiratory tract of a subject, as compared to a control, by at least 1×10^{1.2}, at least 1×10^{1.4}, at least 1×10^{1.5}, at least 1×10^{1.6}, at least 1×10^{1.8}, at least 1×10^{2.0}, at least 1×10^{2.5}, at least 1×10^{3.0}, at least 1×10^{3.5}, at least 1×10^{4.0}, at least 1×10^{4.5}, or at least 1×10^{5.0} TCID₅₀/mL.

The reduction in the amount of the IAV in the lower respiratory tract of a subject administered with the nanoparticles can occur shortly after exposure to, or infection with, the IAV. In some embodiments, the nanoparticle reduces the amount of IAV in the lower respiratory tract (e.g., BAL fluid) of a subject within one, two, three, four, five, six, or seven days of exposure to the IAV. In a particular embodiment, the nanoparticle reduces the amount of IAV in the lower respiratory tract within four days of exposure to the IAV. In some or further embodiments, the nanoparticle further reduces the amount of IAV in the lower respiratory tract within six days of exposure to the IAV. In some embodiments, the amount of IAV in the lower respiratory tract of a subject administered with the nanoparticles is reduced to below clinical detection levels at least one, two, three, four, five, six, seven, or more days earlier than in a subject administered with a control.

The nanoparticles disclosed herein can boost humoral immune responses. For example, the nanoparticles can boost levels of one or more types of antibodies such as, and without limitation, IgA and IgG antibodies. In some embodiments, the nanoparticle elicits an increased amount of IgA antibody in a subject compared to a control. Increased IgA antibody can be determined in relevant samples from a subject, for example serum, mucosa, and/or secretory fluids such as saliva. In a particular embodiment, the nanoparticle increases the amount of IgA antibody in the respiratory mucosa. Increased IgA antibodies can be elicited in any one or more areas of the respiratory mucosa comprising, for example, the upper respiratory tract (determined in, for example, nasal swab), lower respiratory tract (determined in, for example, BAL fluid), lung parenchyma (determined in, for example, lung lysates), or combinations thereof, or in other areas of the respiratory mucosa. In such an embodiment, increased mucosal IgA can facilitate reduced nasal shedding of an IAV and/or enhance clearance of an IAV in a subject.

In some embodiments, the nanoparticle elicits at least 20% more IgA antibody in a subject compared to a control. In some embodiments, the nanoparticle elicits at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 125%, at least 150%, at least 175%, or at least 200% or more IgA antibody in a subject compared to a control.

In some embodiments, the nanoparticle elicits an increased amount of IgG antibody in a subject compared to a control. Increased IgG antibody can be determined in relevant samples from a subject, for example blood, plasma, serum, or lymph.

In some embodiments, the nanoparticle elicits at least 20% more IgG antibody in a subject compared to a control. In some embodiments, the nanoparticle elicits at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 125%, at least 150%, at least 175%, or at least 200% or more IgG antibody in a subject compared to a control.

The subject can be any mammalian subject, for example a human, pig, dog, cow, horse, mouse, rabbit, etc. In some embodiments, the subject is a livestock mammal, particularly a pig. In some embodiments, the subject is a pig at elevated risk for IAV infection, for example by housing in quarters in close proximity to avian livestock (e.g., chickens) which can be or are infected with an avian IAV, or in close proximity to a human which can be or is infected with a human IAV. The subject can be a male or female of any age, size, or other general classifiers.

Optionally, the nanoparticle can be formulated in a medicament. The nanoparticle can be formulated in any suitable medicament including, for example, but not limited to, solids, semi-solids, liquids, and gaseous (inhalant) dosage forms, such as tablets, pills, powders, liquid solutions or suspensions, suppositories, injectables, infusions, inhalants, hydrogels, topical gels, sprays, and the like. Optionally, the medicament comprises a pharmaceutically acceptable excipient/carrier. Optionally, the medicament comprises an effective dose of the inactivated IAV antigen (e.g., a dose effective to prevent or reduce symptoms of an IAV infection).

Also described herein are vaccines comprising an immunogenic composition disclosed herein in an immunogenic carrier, wherein the vaccine is protective against IAV, particularly swine IAV infection. In some embodiments, the vaccine further comprises a pharmaceutically acceptable carrier. The term “immunogenic carrier” as used herein can refer to a first polypeptide or fragment, variant, or derivative thereof which enhances the immunogenicity of a second polypeptide or fragment, variant, or derivative thereof. An “immunogenic carrier” can be fused, to or conjugated/coupled to the desired polypeptide or fragment thereof or provided therewith. See, e.g., European Patent No. EP 0385610 B1, which is incorporated herein by reference in its entirety. An example of an “immunogenic carrier” is mannose conjugated chitosan. In some embodiments the vaccine can comprise mannose conjugated chitosan, inactivated swine IAV antigen, and a pharmaceutically acceptable carrier, wherein the mannose conjugated chitosan encapsulates inactivated swine IAV antigen.

Disclosed are illustrative immunogenic compositions, e.g., vaccine compositions. Additionally, the compositions described herein can comprise one or more immunostimulants. An immunostimulant refers to essentially any substance that enhances or potentiates an immune response (antibody or cell-mediated) to an exogenous antigen. One preferred type of immunostimulant comprises an adjuvant. Many adjuvants contain a substance designed to protect the antigen from rapid catabolism, such as aluminum hydroxide or mineral oil, and a stimulator of immune responses, such as lipid A, Bordetella pertussis or Mycobacterium tuberculosis derived proteins. Certain adjuvants are commercially available as, for example, Freund’s Incomplete Adjuvant and Complete Adjuvant (Difco Laboratories, Detroit, Mich.); Merck Adjuvant 65 (Merck and Company, Rahway, N.J.); AS-2 (GlaxoSmithKline, Philadelphia, Pa.); aluminum salts such as aluminum hydroxide gel (alum) or aluminum phosphate; salts of calcium, iron or zinc; an insoluble suspension of acylated tyrosine; acylated sugars; cationically or anionically derivatized polysaccharides; polyphosphazenes; biodegradable microspheres; monophosphoryl lipid A and quil A. Cytokines, such as GM-CSF, interleukin-2, -7, -12, and other like growth factors, may also be used as adjuvants.

The adjuvant can induce an anti-inflammatory immune response (antibody or cell-mediated). Accordingly, high levels of anti-inflammatory cytokines (anti-inflammatory cytokines may include, but are not limited to, interleukin 4 (IL-4), interleukin 5 (IL-5), interleukin 10 (IL-10), and transforming growth factor beta (TGFβ). Optionally, an anti-inflammatory response can be mediated by CD4+ T helper cells. Bacterial flagellin has been shown to have adjuvant activity (McSorley et al., J. Immunol. 169:3914-19, 2002). Also disclosed are polypeptide sequences that encode flagellin proteins that can be used in adjuvant compositions. Additional adjuvants include but are not limited to, monophosphoryl lipid A (MPL), aminoalkyl glucosaminide 4-phosphates (AGPs), including, but not limited to RC-512, RC-522, RC-527, RC-529, RC-544, and RC-560 (Corixa, Hamilton, Mont.).

In addition, the adjuvant can be one that induces an immune response predominantly of the Th1 type. High levels of Th1-type cytokines (e.g., IFN-γ, TNFα, IL-2 and IL-12) tend to favor the induction of cell mediated immune responses to an administered antigen. In contrast, high levels of Th2-type cytokines (e.g., IL-4, IL-5, IL-6 and IL-10) tend to favor the induction of humoral immune responses. In some embodiments, particularly those in which increased secretion of IgA and IgG antibodies are desirable, the adjuvant can be on in which induces an immune response predominately of the Th2-type. Following application of a vaccine as provided herein, a subject will support an immune response that includes Th1- and Th2-type responses. Optionally, the level of Th1-type cytokines will increase to a greater extent than the level of Th2-type cytokines. The levels of these cytokines may be readily assessed using standard assays. The level of Th2-type cytokines can optionally increase to a greater extent than the level of Th1-type cytokines.

Additional illustrative adjuvants for use in the disclosed compositions (e.g., vaccines) include, for example, a combination of monophosphoryl lipid A, preferably 3-de-O-acylated monophosphoryl lipid A, together with an aluminum salt adjuvants available from Corixa Corporation (Seattle, Wash.; see, for example, U.S. Pat. Nos. 4,436,727; 4,877,611; 4,866,034 and 4,912,094); CpG-containing oligonucleotides (in which the CpG dinucleotide is unmethylated; see e.g., WO 96/02555, WO 99/33488 and U.S. Pat. Nos. 6,008,200 and 5,856,462); immunostimulatory DNA sequences (see e.g., Sato et al., Science 273:352, 1996); saponins such as Quil A, or derivatives thereof, including QS21 and QS7 (Aquila Biopharmaceuticals Inc., Framingham, Mass.); Escin; Digitonin; or Gypsophila or Chenopodium quinoa saponins; Montamide ISA 720 (Seppic, France), SAF (Chiron, Calif., United States), ISCOMS (CSL), MF-59 (Chiron), the SBAS series of adjuvants (e.g., SBAS-2 or SBAS-4, available from GlaxoSmithKline, Philadelphia, Pa.), Detox (Enhanzyn™) (Corixa, Hamilton, Mont.), RC-529 (Corixa, Hamilton, Mont.) and other aminoalkyl glucosaminide 4-phosphates (AGPs), such as those described in pending U.S. patent application Ser. Nos. 08/853,826 and 09/074,720; polyoxyethylene ether adjuvants such as those described in WO 99/52549A1; and combinations thereof. In some embodiments, the adjuvant comprises alpha Galactosylceramide.

In some embodiments, the comparison to a control is statistically significant, as shown by any suitable statistical method.

### Methods of Use

Disclosed herein are methods of reducing transmission of an influenza A virus (IAV) in a subject compared to a control comprising administering to the subject a nanoparticle comprising mannose conjugated chitosan and an inactivated influenza A virus (IAV) antigen, wherein the mannose conjugated chitosan encapsulates the inactivated IAV antigen.

Also disclosed herein are methods of eliciting an immune response against swine influenza A virus in a subject comprising administering to the subject a nanoparticle comprising mannose conjugated chitosan and an inactivated influenza A virus (IAV) antigen, wherein the mannose conjugated chitosan encapsulates the inactivated IAV antigen.

Further disclosed herein are methods of preventing influenza A virus (IAV) infection in a subject compared to a control comprising administering to the subject a nanoparticle comprising mannose conjugated chitosan and an inactivated influenza A virus (IAV) antigen, wherein the mannose conjugated chitosan encapsulates the inactivated IAV antigen.

Also disclosed herein are methods of preventing symptoms of an influenza A virus (IAV) infection in a subject compared to a control comprising administering to the subject a nanoparticle comprising mannose conjugated chitosan and an inactivated influenza A virus (IAV) antigen, wherein the mannose conjugated chitosan encapsulates the inactivated IAV antigen.

Also disclosed herein are methods of increasing specific recall lymphocyte proliferation and cytokines IL-4, IL-10, and/or IFNγ gene expression in a subject compared to a control comprising administering to the subject a nanoparticle comprising mannose conjugated chitosan and an inactivated influenza A virus (IAV) antigen, wherein the mannose conjugated chitosan encapsulates the inactivated IAV antigen.

In any of the methods disclosed herein, the nanoparticle can be any herein disclosed nanoparticle. Similarly, the subject can be any herein disclosed subject, particularly a pig.

In some embodiments, the administering step can include any method of introducing the particle into the subject appropriate for the nanoparticle formulation. In some embodiments, the nanoparticle is administered intranasally. In some embodiments, the nanoparticle is administered in a mist formulation in the nostril of a subject. In some embodiments, at least 1×10⁵ TCID₅₀/mL, at least 1×10⁶ TCID₅₀/mL, at least 1×10⁷ TCID₅₀/mL, or more nanoparticles are administered to the subject. In some embodiments, the administering step includes administering a vaccine comprising any herein disclosed nanoparticle and a pharmaceutically acceptable carrier.

The administering step can include at least one, two, three, four, five, six, seven, eight, nine, or at least ten dosages. The administering step can be performed before the subject exhibits disease symptoms (e.g., vaccination), or as disease symptoms occur. The administering step can be performed prior to, concurrent with, or subsequent to administration of other agents (e.g., adjuvants) to the subject. The administering step can be performed with or without co-administration of additional agents (e.g., immunostimulants).

The nanoparticle can comprise an inactivated IAV antigen which is homologous, heterologous, or heterosubtypic to the IAV of the exposure/infection. In some embodiments, the nanoparticle reduces transmission of, elicits an immune response against, prevents infection of, and/or prevents symptoms of an infection with IAV which is homologous, heterologous, or heterosubtypic to the inactivated IAV antigen comprised in the nanoparticle.

The IAV causing infection can comprise H1N1, H1N2, H2N2, H2N3, H3N1, H3N2, H3N8, H5N1, H5N2, H5N3, H5N6, H5N8, H5N9, H6N1, H7N1, H7N2, H7N3, H7N4, H7N7, H7N9, H9N2, or H10N7 IAV. In some embodiments, the IAV comprises a swine IAV. In some embodiments, the IAV comprises H1N1, H1N2, H3N1, H3N2, H2N3, H4N6, or H9N2 IAV. In some embodiments, the IAV comprises an H1N1, H1N2, or H3N2 IAV. In some embodiments, the subject is infected with more than one IAV, each of which are separately homologous, heterologous, or heterosubtypic to the inactivated IAV antigen comprised in the nanoparticle.

As is typical of many vaccines and prophylactic treatments, the duration of time between the administration step and a subsequent exposure/infection can occasionally affect the outcome of infection in the subject. In some embodiments, the administering step is performed at least three days, at least five days, at least one week, at least two weeks, at least three weeks, or at least one month prior to exposure of the subject to an IAV. In some embodiments, the administering step is performed at about 35 days prior to exposure of the subject to an IAV.

In some embodiments, the methods can comprise a second administration step (e.g., a vaccine boost). A second administration step can, in some embodiments, improve the subject’s immune responses and clinical outcomes upon exposure to an IAV. In some embodiments, the second administration step can be performed at least three days, at least five days, at least one week, at least two weeks, at least three weeks, or at least one month after performing the first administration step.

In some embodiments, the methods can comprise a third, fourth, or fifth, etc. administration step. The nanoparticle of the second or any subsequent administration step can be the same nanoparticle as that of the first administration step, or alternatively, a different herein disclosed nanoparticle.

In some embodiments, the methods reduce nasal shedding of an IAV. The IAV is an infectious IAV and, in some further embodiments, a swine IAV. A sample can be collected from the upper respiratory tract of a subject by any suitable method (e.g., by nasal swab of shedding mucous).

The amount of reduction of IAV in the upper respiratory tract of a subject administered with the nanoparticles can be quantified by any suitable method. In some embodiments, the amount of reduction of IAV in the upper respiratory tract of a subject is quantifiable by TCID₅₀/mL.In some embodiments, the methods reduce the amount of the IAV in an upper respiratory tract of a subject by at least 1×10¹ TCID₅₀/mL compared to a control. In some embodiments, the methods reduce the amount of the IAV in an upper respiratory tract of a subject, as compared to a control, by at least 1×10^{1.2}, at least 1×10^{1.4}, at least 1×10^{1.5}, at least 1×10^{1.6}, at least 1×10^{1.8}, at least 1×10^{2.0}, at least 1×10^{2.5}, at least 1×10^{3.0}, at least 1×10^{3.5}, at least 1×10^{4.0}, at least 1×10^{4.5}, or at least 1×10^{5.0} TCID₅₀/mL.

In some embodiments, the methods reduce the amount of the IAV in the upper respiratory tract of a subject shortly after exposure to, or infection with, the IAV. In some embodiments, the methods reduce the amount of IAV in the upper respiratory tract of a subject within one, two, three, four, five, six, or seven days of exposure to the IAV. In a particular embodiment, the methods reduce the amount of IAV in the upper respiratory tract within four days of exposure to the IAV. In some or further embodiments, the methods further reduce the amount of IAV in the upper respiratory tract within six days of exposure to the IAV. In some embodiments, the methods reduce the levels of IAV in a subject to below clinical detection levels at least one, two, three, four, five, six, seven, or more days earlier than in a subject administered with a control. In some embodiments, the methods prevent an increase in the amount of infecting IAV to a level which causes symptoms of the infection (e.g., flu-like symptoms).

In some embodiments, the methods reduce the amount of IAV in the lower respiratory tract. Reduced IAV levels can be determined in a sample collected from the lower respiratory tract of a subject by any suitable method (e.g., in BAL fluid). In some embodiments, the reduced amount of an IAV in the lower respiratory tract can be in addition to reduced nasal shedding of the IAV.

The amount of reduction of IAV in the lower respiratory tract of a subject administered with the nanoparticles can be quantified by any suitable method (e.g., by TCIDso/mL). In some embodiments, the methods reduce the amount of the IAV in the lower respiratory tract (e.g., BAL fluid) of a subject by at least 1×10¹ TCID₅₀/mL compared to a control. In some embodiments, the methods reduce the amount of the IAV in an upper respiratory tract of a subject, as compared to a control, by at least 1×10^{1.2}, at least 1×10^{1.4}, at least 1×10^{1.5}, at least 1×10^{1.6}, at least 1×10^{1.8}, at least 1×10^{2.0}, at least 1×10^{2.5}, at least 1×10^{3.0}, at least 1×10^{3.5}, at least 1×10^{4.0}, at least 1×10^{4.5}, or at least 1×10^{5.0} TCID₅₀/mL.

In some embodiments, the methods reduce the amount of the IAV in the lower respiratory tract of a subject administered with the nanoparticles shortly after exposure to, or infection with, the IAV. In some embodiments, the methods reduce the amount of IAV in the lower respiratory tract (e.g., BAL fluid) of a subject within one, two, three, four, five, six, or seven days of exposure to the IAV. In a particular embodiment, the methods reduce the amount of IAV in the lower respiratory tract of a subject within four days of exposure to the IAV and, in some or further embodiments, reduce the amount of IAV within six days of exposure to the IAV. In some embodiments, the methods reduce the amount of IAV in the lower respiratory tract to below clinical detection levels at least one, two, three, four, five, six, seven, or more days earlier than in a subject administered with a control.

The methods disclosed herein can boost humoral immune responses. For example, the methods can boost levels of one or more types of antibodies such as, and without limitation, IgA and IgG antibodies. In some embodiments, the methods increase the amount of IgA antibody in a subject. Increased IgA antibody can be determined in relevant samples from a subject, for example serum, mucosa, and/or secretory fluids such as saliva. In a particular embodiment, the methods elicit an increased amount of IgA antibody, as compared to a control, in the respiratory mucosa. For example, the methods increase IgA antibodies in any one or more areas of the respiratory mucosa comprising the upper respiratory tract (determined in, for example, nasal swab), lower respiratory tract (determined in, for example, BAL fluid), lung parenchyma (determined in, for example, lung lysates), or combinations thereof, or in other areas of the respiratory mucosa. In such embodiments, increased mucosal IgA can facilitate reduced nasal shedding of an IAV and/or enhance clearance of an IAV in a subject.

In some embodiments, the methods elicit at least 20% more IgA antibody in a subject. In some embodiments, the methods elicit at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 125%, at least 150%, at least 175%, or at least 200% or more IgA antibody in a subject compared to a control.

In some embodiments, the methods elicit an increased amount of IgG antibody in a subject. Increased IgG antibody can be determined in relevant samples from a subject, for example blood, plasma, serum, or lymph. In some embodiments, the methods elicit at least 20% more IgG antibody in a subject compared to a control. In some embodiments, the methods elicit at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 125%, at least 150%, at least 175%, or at least 200% or more IgG antibody in a subject compared to a control.

In some embodiments, the methods enhance cell-mediated immune responses to an IAV in a subject. In some embodiments, the methods elicit increased amounts of IFNγ in a subject compared to a control. Increased amounts of IFNγ can be determined in relevant samples from a subject, for example blood, plasma, serum, or lymph. In some embodiments, the methods elicit at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 125%, at least 150%, at least 175%, at least 200%, or more IFNγ in a subject compared to a control. In some embodiments, the methods elicit at least 2-fold, at least 3-fold, at least 4-fold, at least 5-fold, at least 10-fold, at least 15-fold, at least 20-fold, or more IFNγ in a subject compared to a control. In some embodiments, the methods elicit increased amounts of IFNγ in a subject after administering the nanoparticles but before exposure to an IAV. In some or further embodiments, the methods elicit increased amounts of IFNγ in a subject after administering the nanoparticles and after exposure to an IAV.

In some embodiments, the methods can elicit enhanced helper T cell and memory T cell frequencies in a subject. Enhanced helper T cell and memory T cell frequency can be determined in relevant samples from a subject, for example blood, plasma, serum, or lymph. In some embodiments, the helper and memory T cells having enhanced frequency comprise CD3⁺CD4⁺CD8α⁺ cells. In some embodiments, the methods elicit at least 5%, at least 10%, at least 15%, or at least 20% more helper and memory T cells as a percentage of CD3⁺ cells in a subject compared to a control. In some embodiments, the methods elicit enhanced helper and memory T cell frequencies after exposure to an IAV.

In some embodiments, the methods elicit enhanced cytotoxic T cell (CTL) frequencies in a subject. Enhanced CTL frequency can be determined in relevant samples from a subject, for example blood, plasma, serum, or lymph. In some embodiments, the CTLs having enhanced frequency comprise CD3⁺CD4⁻CD8αβ⁺ cells. In some embodiments, the methods elicit at least 5%, at least 10%, at least 15%, or at least 20% more CTL as a percentage of CD3⁺ cells in a subject compared to a control.

In some embodiments, the methods reduce tissue damage in the lungs of a subject caused by subsequent IAV infection. The damage can be, for example, pneumonic lesions, microscopic lesions, or other IAV-induced tissue damage in the lungs. Reduced damage can be measured and demonstrated by, for example and without limitation, reduced percentage of lung consolidation, or by reduced interstitial pneumonia in lung tissue (e.g., in H&E stained tissue).

In some embodiments, the methods can reduce levels of pro-inflammatory cytokines in a subject. Reduced levels of pro-inflammatory cytokines can, in some embodiments, occur systemically (e.g., measured in serum), or can be measured locally in BAL fluid. In some embodiments, the pro-inflammatory cytokine comprises IL-6.

In some embodiments, the methods further comprise administering any one or more of the herein disclosed immunostimulants (e.g., adjuvants). The immunostimulant can be administered prior to, concurrent with, or subsequent to administration of the nanoparticles.

In some embodiments, the nanoparticle can be provided in the form of a medicament or vaccine. The medicament or vaccine can further comprise a pharmaceutically acceptable excipient/carrier.

In some embodiments, the comparison to a control in any method is statistically significant, as shown by any suitable statistical method.

### Kits

Also disclosed herein are kits comprising a nanoparticle comprising mannose conjugated chitosan and an inactivated influenza A virus (IAV) antigen, wherein the mannose conjugated chitosan encapsulates the inactivated IAV antigen, and a device for intranasal administration.

In any of the kits disclosed herein, the nanoparticle can be any herein disclosed nanoparticle. Similarly, the kit can be used to administer the nanoparticle to any herein disclosed subject, particularly a pig.

The kit comprises a device for intranasal administration. The device for intranasal administration, in some embodiments, is configured for insertion into the nostril of a subject. In some embodiments, the device provides a mist of liquid formulation comprising the nanoparticle. In some embodiments, the device provides a mist of a vaccine comprising the nanoparticle and a pharmaceutically acceptable carrier. In some embodiments, the device is a nasal spray device.

### EXAMPLES

To further illustrate the principles of the present disclosure, the following examples are put forth so as to provide those of ordinary skill in the art with a complete disclosure and description of how the compositions, articles, and methods claimed herein are made and evaluated. They are intended to be purely exemplary of the invention and are not intended to limit the scope of what the inventors regard as their disclosure. These examples are not intended to exclude equivalents and variations of the present invention which are apparent to one skilled in the art. Unless indicated otherwise, temperature is °C or is at ambient temperature, and pressure is at or near atmospheric. There are numerous variations and combinations of process conditions that can be used to optimize product quality and performance. Only reasonable and routine experimentation will be required to optimize such process conditions.

### Example 1. Immunity and Protective Efficacy of Mannose Conjugated Chitosan-Based Influenza Nanovaccine in Maternal Antibody Positive Pigs

Parenteral administration of killed/inactivated swine influenza A virus (SwIAV) vaccine in weaned piglets provides variable levels of immunity due to the presence of preexisting virus specific maternal derived antibodies (MDA). To overcome the effect of MDA on SwIAV vaccine in piglets, an intranasal deliverable killed SwIAV antigen (KAg) encapsulated chitosan nanoparticles called chitosan-based NPs encapsulating KAg (CS NPs-KAg) vaccine was developed. Further, to target the vaccine to dendritic cells and macrophages which express mannose receptor, mannose was conjugated to chitosan (mCS) and KAg encapsulated mCS nanoparticles called mannosylated chitosan-based NPs encapsulating KAg (mCS NPs-KAg) vaccine were formulated. In MDA-positive piglets, prime-boost intranasal inoculation of mCS NPs-KAg vaccine elicited enhanced homologous (H1N2-OH10), heterologous (H1N1-OH7), and heterosubtypic (H3N2-OH4) influenza virus-specific secretory IgA (sIgA) antibody response in nasal passage compared to CS NPs-KAg vaccinates. In vaccinated upon challenged with a heterologous SwIAV H1N1, both mCS NPs-KAg and CS NPs-KAg vaccinates augmented H1N2-OH10, H1N1-OH7, and H3N2-OH4 virus-specific sIgA antibody responses in nasal swab, lung lysate, and bronchoalveolar lavage (BAL) fluid; and IgG antibody levels in lung lysate and BAL fluid samples. Whereas the multivalent commercial inactivated SwIAV vaccine delivered intramuscularly increased serum IgG antibody response. In mCS NPs-KAg and CS NPs-KAg vaccinates increased H1N2-OH10 but not H1N1-OH7 and H3N2-OH4-specific serum hemagglutination inhibition titers were observed. Additionally, mCS NPs-KAg vaccine increased specific recall lymphocyte proliferation and cytokines IL-4, IL-10, and IFNγ gene expression compared to CS NPs-KAg and commercial SwIAV vaccinates in tracheobronchial lymph nodes. Consistent with the immune response both mCS NPs-KAg and CS NPs-KAg vaccinates cleared the challenge H1N1-OH7 virus load in upper and lower respiratory tract more efficiently when compared to commercial vaccine. The virus clearance was associated with reduced gross lung lesions. Overall, mCS NP-KAg vaccine intranasal immunization in MDA-positive pigs induced a robust cross-reactive immunity and offered protection against influenza virus.

### Background

Swine influenza is an acute respiratory disease of pigs caused by swine influenza A virus (SwIAV) (Schultz-Cherry et al., 2013). Pigs are naturally vulnerable to IAV-associated with secondary bacterial infections (Sunwoo et al., 2018). Swine IAV is an economic threat to the global pig industry (Pomorska-Mól et al., 2017). Commonly circulating SwIAV strains in swine population are H1N1, H1N2, and H3N2 (Vincent et al., 2008). In the United States, periodically human infections are occurred from some of the SwIAVs (Karasin et al., 2000). In last two decades, triple reassortant SwIAVs have been isolated from pigs (Karasin et al., 2000), and its association with human infections have also been documented (Gray et al., 2007; Newman et al., 2008). The most recent is the 2009 pandemic H1N1 SwIAV spillover to humans (Mastin et al., 2011;Trebbien et al., 2011). Therefore, vaccination of pigs is a common practice to reduce the influenza burden in swine industry and to avoid the risk of zoonotic transmission to humans (Thomas et al., 2010). The SwIAV vaccine inoculated into sows protects the herd from infection and heightens the transfer of maternally-derived antibodies (MDA) to offspring through colostrum (Deblanc et al., 2018;Chamba Pardo et al., 2019). However, a number of studies have revealed that MDA offered various levels of protection against IAV infection in piglets (Deblanc et al., 2018;Sunwoo et al., 2018;Chamba Pardo et al., 2019). In weaned piglets, MDA interferes with parenteral administered killed/inactivated influenza virus vaccines, resulting in poor induction of antibody responses and documented evidence of vaccine-associated enhanced respiratory disease (Markowska-Daniel et al., 2011;Vincent et al., 2012;Sandbulte et al., 2014;Sunwoo et al., 2018).

The MDA inhibits the vaccine-induced IgG antibody and does not interfere with the secretory IgA (sIgA) antibody production (Zhang et al., 2016). Intranasally (IN) administered inactivated IAV vaccine in mice overcomes the MDA interference and provides complete protection in offspring (Zhang et al., 2016). Influenza viruses use nasal mucosa as a main entry site. Effective vaccines delivered IN trigger the mucosal immunity and offer the frontline defense against the infection (Cox et al., 2004). Further, IN vaccination activates the B and T cells in the nasal-associated lymphoid tissues and induce specific antibody and cell-mediated immune responses.

Chitosan is a biocompatible polymer, and its protonated positively charged amino groups electrostatically interact with negative charged mucus sialic acid and epithelial surfaces to become mucoadhesive vehicle (M Ways et al., 2018;Renu et al., 2020d). Hence, chitosan nanoparticles (CS NPs) were used as a mucosal vaccine delivery carrier for the poultry and swine vaccines to combat infectious diseases (Akerele et al., 2020;Han et al., 2020;Renu et al., 2020a;Renu et al., 2020c). In protein antigens encapsulated CS NPs, treated immune cells in vitro demonstrate upregulated multiple Toll-like receptors (TLRs), Th1 and Th2 cytokines gene expression (Renu et al., 2020c). In SwIAV killed antigen loaded CS NPs treated dendritic cells (DCs) observed enhanced secretion of innate, pro-inflammatory and Th1 cytokines, and in IN vaccinated pigs, induction of enhanced cross-reactive mucosal immunity has been observed (Dhakal et al., 2018).

The calcium-dependent (C-type) lectin family mannose receptor (MR) is a carbohydrate binding protein, primarily expressed by the DCs and macrophages (Apostolopoulos et al., 2013). The MR binds to mannosylated protein and the antigens uptaken through MR are efficiently processed and presented through major histocompatibility pathways by DCs (Engering et al., 1997;Apostolopoulos et al., 2013). Mannose ligand is internalized by DCs through receptor-mediated endocytosis (Shi et al., 2017). In vitro, mannan ligand-coated nanoparticles readily binds to MR expressing cells and internalized (Cui et al., 2003). Mannose ligand in mannosylated CS NPs interacts with MR on the surface of macrophages and facilitates its uptake (Peng et al., 2015). In vivo, glycosylated nanoparticles rapidly shuttle to the follicular DCs network and are concentrated in germinal centers of lymph nodes thereby triggering the innate immune-mediated recognition pathway and promotes antigen-specific responses (Tokatlian et al., 2019). For these reasons, the MR receptor on cells is a possible target for vaccine delivery (Apostolopoulos et al., 2013). Mannose ligand was conjugated with chitosan (mCS) and killed SwIAV antigen (KAg) encapsulated mCS NPs (mCS NPs-KAg) vaccine was formulated. The efficacy of IN-administered mCS NPs-KAg and KAg encapsulated CS NPs (CS NPs-KAg) vaccine in MDA-positive pigs were determined and compared to an intramuscularly (IM) administered multivalent commercial SwIAV vaccine as a positive control.

### Materials and Methods

### Preparation of Influenza Viruses and Source of Commercial SwIAV Vaccine

The field isolates of IAVs - A/Swine/OH/FAH10-⅒ (H1N2-OH10), A/Swine/OH/24366/2007 (H1N1-OH7), and A/Turkey/OH/313053/2004 (H3N2-OH4) were grown in Madin-Darby Canine Kidney epithelial (MDCK) cells (Dhakal et al., 2017). The virus-rich cell free supernatant was clarified using sucrose density gradient ultracentrifugation and the viral pellet was suspended in phosphate-buffered saline (PBS). Viruses were inactivated using binary ethyleneimine and the inactivation efficiency was confirmed by re-culture in MDCK cells (Dhakal et al., 2017) and as henceforth it is called KAg. The virus titers were analyzed in MDCK cells (Dhakal et al., 2017). The protein content in inactivated virus was tested using a micro-BCA protein assay kit (Thermo Scientific, MA, USA) as per the company recommendations. Commercial inactivated SwIAV vaccine (FluSure XP®) was obtained from Zoetis (MI, USA) and used as per the company recommendations. The FluSure XP® is a multivalent vaccine containing H1N1, H1N2, and H3N2 SwIAVs.

### Formulation of Experimental Vaccines

Chitosan-based NPs encapsulating KAg (CS NPs-KAg) and mannosylated chitosan-based NPs encapsulating KAg (mCS NPs-KAg) vaccines were prepared using an ionic gelation method as previously described (Renu et al., 2020a). For mannose-conjugated chitosan (mCS) preparation, 40 mg each of mannose (Sigma, MO) and sodium triacetoxyborohydride (Sigma, MO) mixture in 0.2 M borate buffer was slowly added into 200 mg chitosan [1% (w/v)] suspension (Renu et al., 2020c) under magnetic stirring for 72 h at 56° C. (Chaubey and Mishra, 2014). The mCS was dialyzed against milli-Q-water for 48 h. Both chitosan and mannose modified chitosan were dissolved in 1% acetic acid solution. Twenty milligrams of mCS or chitosan were added into 20 ml milli-Q-water under magnetic stirring, pH was adjusted to 4.3 and mixed with 2 mg KAg (H1N2-OH10) in 3-(N-morpholino) propanesulfonic acid (MOPS) buffer pH 7.4. Followed by tripolyphosphate [1% (w/v) (Sigma, MO)] 5 mg in 10 ml milli-Q-water was added dropwise and the mCS NPs-KAg or CS NPs-KAg vaccines were obtained after centrifugation at 10.976 × g for 30 minutes, washed, dispersed in milli-Q-water and used for vaccination. Both the vaccines had approximately 80% antigen encapsulation efficiency and characterized as described previously (Renu et al., 2020a;Renu et al., 2020c). Both the chitosan-based vaccine formulations were freshly prepared and used in animals.

### Experimental Plan

Three genetically related pregnant sows (Yorkshire x Landrace) were vaccinated with FluSure XP® vaccine at 2 and 5 weeks before farrowing as per the manufacturer’s instruction, and the naturally born piglets were weaned at three weeks of age and transported to the Ohio Agricultural Research and Development Center (OARDC) biosafety level-2 animal holding facility. From each sow litter received over 60% piglets which were well grown and looked very healthy and randomized to have at least one piglet from each sow present in every experimental group. Blood samples from piglets were collected and tested for SwIAV-specific antibody titers. The MDA-positive piglets (n=19) were randomly distributed into a five experimental groups as follows: (i) Mock (no vaccination and no challenge, n=3); (ii) Mock-challenge (no vaccination and challenge, n=4); (iii) FluSure XP® vaccine (n=4); (iv) CS NPs-KAg vaccine (10⁷ TCID₅₀ equivalent of KAg from H1N2-OH10 virus to each piglet, n=4); and (v) mCS NPs-KAg vaccine (10⁷ TCID₅₀ equivalent of KAg from H1N2-OH10 virus to each piglet, n=4).

Experimental piglets at age three weeks received the mCS NPs-KAg or CS NPs-KAg vaccine through both the nostrils using a spray mist delivery device (Prima Tech USA, NC). Commercial FluSure XP® vaccine was administered IM as per the company recommendation. Pigs received a booster dose of vaccine like the prime dose three weeks later. Two weeks after booster vaccination, experimental vaccinates (except mock control group) were challenged (Ch) with a heterologous H1N1-OH7 virus 6×10⁶ TCID₅₀ (50% IN and 50% intratracheal after anaesthetizing the animals) (Dhakal et al., 2017). Pigs were monitored daily for clinical signs (Renu et al., 2020b) and euthanized 6 days after challenge by anesthetizing followed by exsanguination. Blood and nasal swab samples were collected before and after prime and boost vaccinations. At necropsy, along with blood and nasal swab samples, bronchoalveolar lavage (BAL) fluid, lung samples for preparing lung lysate (represents lung parenchyma), tracheobronchial lymph nodes (TBLN) in DMEM for isolating mononuclear cells (MNCs) and pieces of TBLN tissues in RNAlater were collected, processed and stored as described previously (Dhakal et al., 2017). Gross lung lesions were scored based on the presence of virus affected purple red consolidation in each lung lobe. The final lung lesion score of each pig was obtained by averaging all the scores recorded in dorsal and ventral lobes. Images of dorsal and ventral views of the lungs were captured from all the pigs.

### Antibody Titration

The pre-titrated KAg extracted from H1N2-OH10, H1N1-OH7, or H3N2-OH4 viruses were coated in duplicate (Renu et al., 2020b) in 96-well plates (Greiner bio-one, Monroe, NC, USA) and incubated overnight at 4° C. Plates were washed with PBS Tween-20 (0.05%) (PBST) and blocked with 5% skim milk powder in PBST for 2 h, at room temperature (RT). After plates washed, serially diluted nasal swab, lung lysate and BAL fluid samples were analyzed for sIgA; and serum, lung lysate and BAL fluid samples for IgG antibodies by adding to marked duplicate wells and incubated overnight at 4° C. Plates were washed and horseradish peroxidase conjugated goat anti-pig IgA (Bethyl Laboratories, Montgomery, TX) or goat anti-pig IgG (KPL, Gaithersburg, MD) antibodies were added and incubated for 2 h, at RT. Plates were washed and 1:1 mixture of peroxidase substrate solution B and TMB peroxidase substrate (KPL, Gaithersburg, MD) was added, and after 10-20 minutes the reaction was terminated with 1 M phosphoric acid solution. The optical density (OD) values were measured at 450 nm in ELISA Spectramax microplate reader (Molecular devices, CA), and samples corrected OD values were attained after subtraction of the blank value.

Hemagglutination inhibition antibody titers in serum samples collected at day 6 post challenge against H1N2-OH10, H1N1-OH7, and H3N2-OH4 viruses were analyzed as reported earlier (Hiremath et al., 2016). Briefly, tenfold serially diluted heat inactivated sera in triplicates was added to eight HA units of virus and incubated at 37° C. for 1 h. The hemagglutination inhibition titers were calculated by using the 50 % endpoint method.

### Cell Proliferation Analysis

The isolated peripheral blood mononuclear cells (PBMCs) at day post vaccination 35, and TBLN MNCs isolated at day post challenge (DPC)-6 were subjected to cell proliferation and flow cytometry analyses as reported previously (Dhakal et al., 2017;Renu et al., 2020b). Briefly, 1×10⁶ cells/well in triplicate in 10% FBS containing RPMI medium was plated in a 96 well flat-bottom plate (Greinerbio-one, NC). Cells were either unstimulated or stimulated with 0.1 multiplicity of infection (MOI) vaccine (H1N2-OH10) and challenge (H1N1-OH7) viruses for 72 h at 37° C. in 5% CO₂ incubator. The 20 µl MTS + PMS solution (Promega, WI) was added to each well before 4 h of 72 h incubation, and the OD at 490 nm was recorded using the ELISA Spectramax microplate reader. Stimulation index was calculated by dividing OD of stimulated from OD of unstimulated cells of the same animal.

### Quantitative Reverse Transcription PCR (qRT-PCR) Analysis

Total RNA was extracted from TBLN stored in RNAlater using TRIzol reagent (Invitrogen, Carlsbad, CA). The cDNA syntheses (Renu et al., 2020c) was attained from 2 µg of total RNA (Renu et al., 2018), and the target cytokine IL-4, IL-10 and IFNγ, and internal control β-actin (FIG. 8) (Gao et al., 2014;Renu et al., 2020a) genes expression were achieved using the SYBR Green Supermix kit (Bio-Rad Laboratories, CA) by qRT-PCR (Applied Biosystems, CA). Gene expression in fold changes was calculated as described (Renu et al., 2018).

### Challenge Virus Titration

The procedure for virus titration was followed as described previously (Dhakal et al., 2017). Briefly, nasal swab collected at DPC-4 and DPC-6, BAL fluid and lung lysate samples collected at DPC-6 were tenfold serially diluted in TPCK-trypsin containing serum-free DMEM medium, added into monolayer of MDCK cells and incubated for 36 h at 37° C. in 5% CO₂ incubator. Cells were fixed and immunostained with IAV nucleoprotein specific primary antibody (CalBioreagents, CA) followed by AlexaFluor 488 conjugated goat anti-mouse IgG (H+L) secondary antibody (Life Technologies, CA). Immunofluorescence signal was observed in a fluorescent microscope (IX51, Olympus, Tokyo, Japan) and the virus titers were calculated.

### Statistical Analyses

Two-way ANOVA followed by a Bonferroni test was used for statistical analyses of ELISA data using the GraphPad Prism 8 (GraphPad Software, Inc., CA). The remaining experimental data were examined by one-way ANOVA followed by Tukey’s post-hoc comparison test. Date were presented as mean of three to four pigs ± standard error mean (SEM) of each experimental group. Results were considered statistically significant when p < 0.05.

### Results

### Prime-Boost Immunization of MDA-Positive Pigs With mCS NPs-KAg Vaccine Prior to Challenge Increased the Cross-reactive sIgA Antibody Response

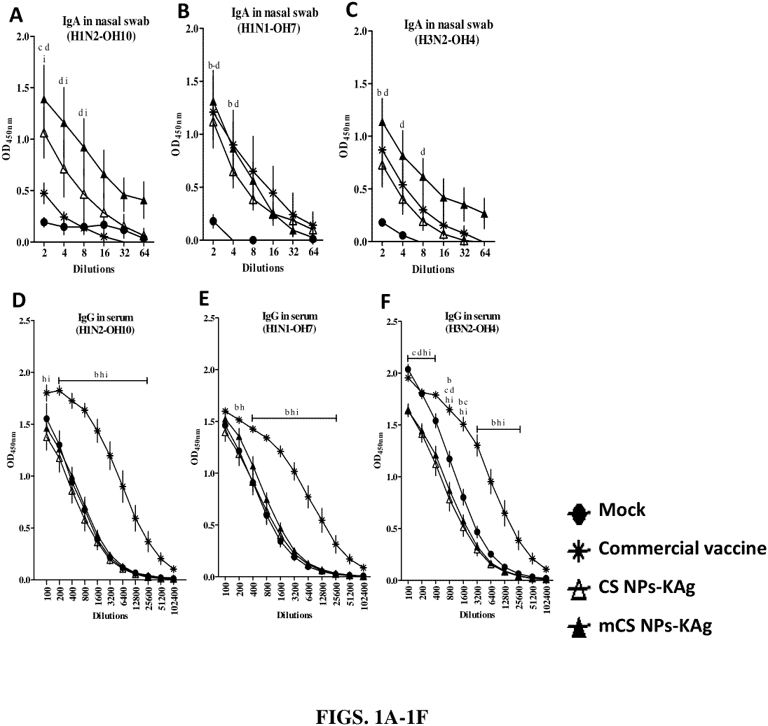

All the weaned piglets born to vaccinated mothers used in this experimental trial had high levels of SwIAV specific MDA in serum, with no significant difference between the groups (FIG. 8). In mCS NPs-KAg vaccine inoculated MDA-positive pigs after two doses of vaccination at day post vaccination 35 detected enhanced homologous (H1N2-OH10) and heterosubtypic (H3N2-OH4) IAV-specific sIgA antibody levels in nasal swabs at all the tested dilutions compared to mock, commercial and CS NPs-KAg vaccinates (FIGS. 1A, C). Compared to commercial SwIAV vaccine, both mCS NPs-KAg and CS NPs-KAg vaccinates increased H1N2-OH10 virus-specific sIgA antibody levels in nasal swabs, while mCS NPs-KAg vaccine increased level was significantly (p < 0.05) higher (FIG. 1A). mCS NPs-KAg and commercial vaccinates had significantly (p < 0.05) increased H1N1-OH7 and H3N2-OH4 viruses-specific sIgA antibody levels in nasal swab compared to mock pigs (FIGS. 1B, C). On the other hand, commercial SwIAV vaccinates significantly (p < 0.05) increased H1N2-OH10, H1N1-OH7, and H3N2-OH4 viruses-specific IgG antibody levels in serum than all the other groups were observed (FIGS. 1D-F).

### mCS NPs-KAg and CS NPs-KAg Vaccinates Augmented Cross-reactive sIgA Antibody Response Following Challenge Infection

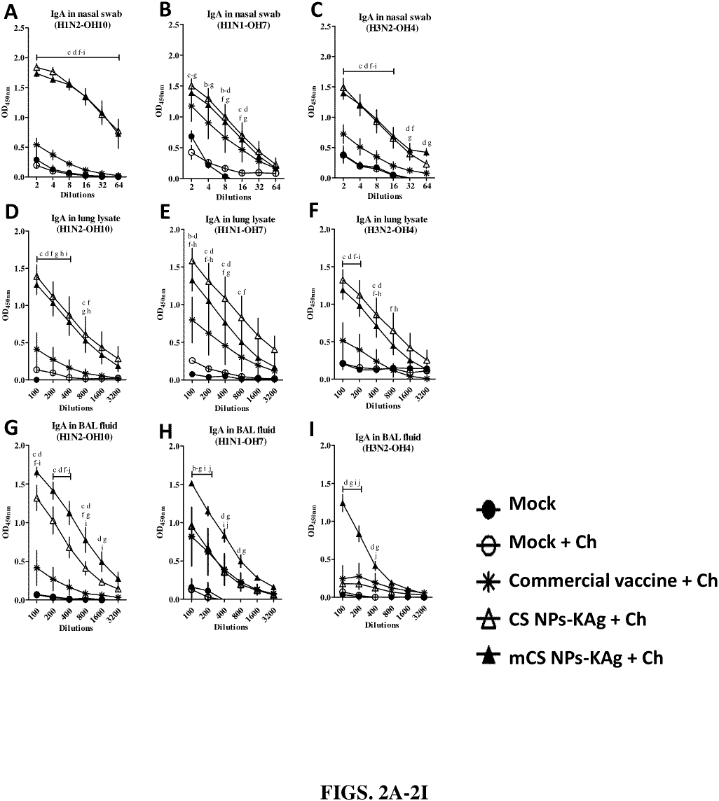

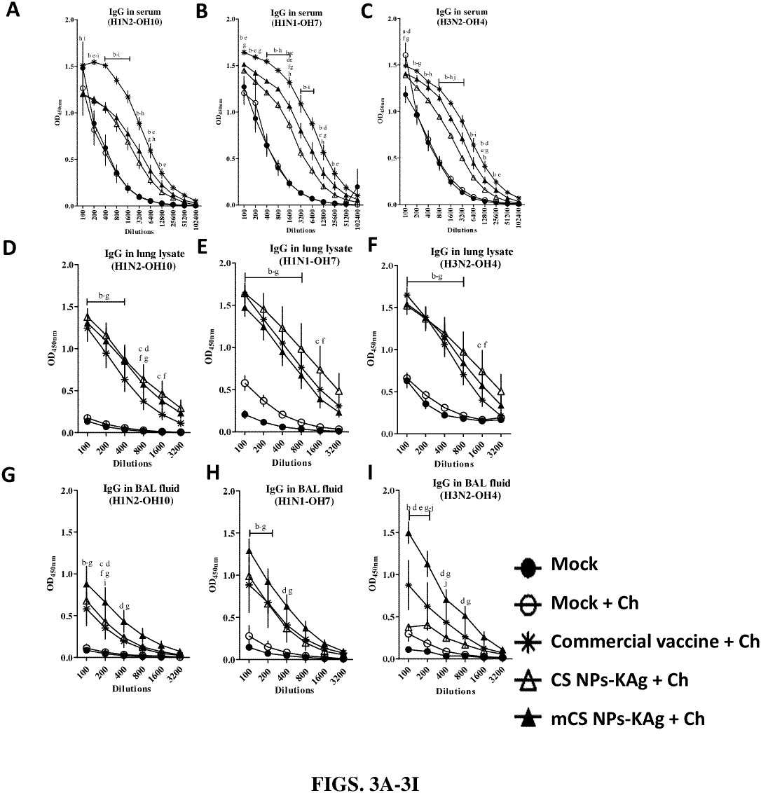

In SwIAV challenged pigs, mCS NPs-KAg and CS NPs-KAg vaccinates had a significantly (p < 0.05) higher H1N2-OH10 and H3N2-OH4 virus-specific sIgA antibody levels in nasal swab at tested all the dilutions (2 to 64) compared to other groups including commercial influenza vaccine (FIGS. 2A, C). Although, in mCS NPs-KAg and CS NPs-KAg vaccinates increased H1N1-OH7 virus specific sIgA antibody levels in nasal secretions was detected compared to other experimental groups, statistical significance (p < 0.05) was reached compared to the mock and mock-challenge groups (FIG. 2B). As in the nasal swab samples, the H1N2-OH10 and H3N2-OH4 viruses-specific sIgA antibody levels in lung lysate was also significantly (p < 0.05) augmented by mCS NPs-KAg and CS NPs-KAg vaccines compared to all the other groups (FIGS. 2D, F). In addition, unlike the sIgA level in the nasal cavity, the CS NPs-KAg vaccinates had significantly (p < 0.05) increased H1N1-OH7 virus specific sIgA antibody levels in lung lysates compared to commercial vaccine (FIG. 2E). The H1N2-OH10 virus specific sIgA antibody level in BAL fluid was significantly (p < 0.05) increased in both mCS NPs-KAg and CS NPs-KAg vaccinates over other groups including commercial vaccine received animals (FIG. 2G). It is important to note that compared to all the groups including CS NPs-KAg vaccinates, the mCS NPs-KAg vaccinates had a remarkably (p < 0.05) increased H1N2-OH10, H1N1-OH7, and H3N2-OH4 virus-specific sIgA antibody levels in BAL fluid (FIGS. 2G-I).

### mCS NPs-KAg and CS NPs-KAg Vaccinates Had Increased Cross-reactive IgG Antibody Response After Challenge Infection in Serum, Lung Lysate and BAL Fluid

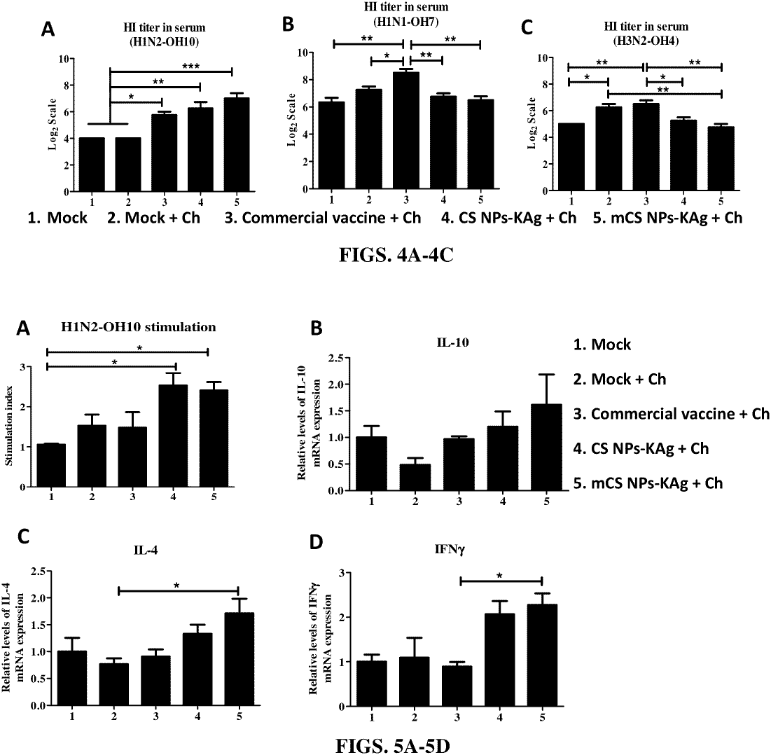

In the serum of mCS NPs-KAg, CS NPs-KAg and commercial SwIAV vaccinates, significantly (p < 0.05) increased IgG antibody levels specific to H1N2-OH10, H1N1-OH7 and H3N2-OH4 viruses compared to mock and mock-challenge groups was observed. The commercial flu vaccine received animals had significantly (p < 0.05) increased IgG antibody levels compared to CS NPs-KAg and mCS NPs-KAg vaccinates (FIGS. 3A-C). In CS NPs-KAg, mCS NPs-KAg and commercial vaccinates a significant (p < 0.05) increase in H1N2-OH10, H1N1-OH7 and H3N2-OH4 viruses specific IgG antibody levels in lung lysate sample compared to mock and mock-challenge groups was evident (FIGS. 3D-F). Compared to pigs given commercial vaccine in CS NPs-KAg vaccinates detected numerically increased H1N2-OH10, H1N1-OH7, and H3N2-OH4 viruses specific IgG antibody levels in lung lysates (FIGS. 3D-F). In the BAL fluid, like the observed enhanced sIgA antibody, the IgG antibody levels in mCS NPs-KAg vaccinates was also significantly (p < 0.05) increased against H3N2-OH4, H1N2-OH10, and H1N1-OH7 viruses at some of the tested dilutions compared to commercial vaccine (FIGS. 3G-I). Furthermore, in mCS NPs-KAg vaccinates, significantly (p < 0.05) increased H3N2-OH4 virus specific IgG antibody level in BAL fluid was detected when these values were compared to values obtained from the BAL fluids of CS NPs-KAg vaccinates (FIG. 3I).

### Hemagglutination Inhibition (HI) Titers in the Serum of mCS NPs-KAg and CS NPs-KAg Vaccinates were Increased Against the Vaccine Virus but Not Against Variant IAVs

The mCS NPs-KAg (p < 0.001), CS NPs-KAg (p < 0.01) and commercial vaccines (p < 0.05) significantly increased the H1N2-OH10 virus specific HI titers in serum compared to values obtained in mock and mock-challenge pigs (FIG. 4A). Compared to commercial vaccine, both mCS NPs-KAg and CS NPs-KAg vaccinates had increased serum HI titers against H1N2-OH10 virus by 2.3 and 1.4 times, respectively (FIG. 4A). Commercial vaccine received animals had significantly higher H1N1-OH7 (p < 0.01) and H3N2-OH4 (p < 0.05) viruses specific HI titers compared to mCS NPs-KAg and CS NPs-KAg vaccinates (FIGS. 4B, C). In addition, commercial vaccine induced significantly higher H1N1-OH7 virus specific HI titers than those recorded in mock (p < 0.01) and mock-challenge (p < 0.05) pig groups (FIG. 4B). Both the commercial vaccine (p < 0.01) and mock-challenge (p < 0.05) pigs had a significantly increase HI titers against H3N2-OH4 virus compared to mock group. In addition, mock-challenge group had increased (p < 0.01) tires compared to mCS NPs-KAg group (FIG. 4C).

### mCS NPs-KAg and CS NPs-KAg Vaccines Increased the Cell-mediated Immunity in The Local Cytokine Gene Expression in TBLN

The mCS NPs-KAg and CS NPs-KAg vaccines induced a virus-specific proliferative cellular immune response in TBLN MNCs at DPC-6 as determined by analyzing the lymphocyte proliferation index (FIG. 5A). The vaccine (H1N2-OH10) virus stimulated lymphocyte stimulation index in TBLN of both the mCS NPs-KAg and CS NPs-KAg vaccinates was significantly (p < 0.05) higher compared to mock group (FIG. 5A). However, the vaccinated experimental groups did not have significant increase in challenge (H1N1-OH7) virus specific lymphocyte stimulation index values. The different cytokine gene expression profiles as correlates with the mCS NPs-KAg and CS NPs-KAg vaccines enhanced humoral and cell mediated immune responses were analyzed. Both the mCS NPs-KAg and CS NPs-KAg vaccinates had increased expression of Th2 (IL-4), Th1 (IFNγ) and IL-10 cytokine mRNA levels compared to all the other experimental groups including commercial vaccine (FIGS. 5B-D). While only in mCS NPs-KAg vaccinates the upregulated IL-4 and IFNγ cytokine gene expressions were significantly (p < 0.05) higher compared to mock-challenge and commercial vaccine groups, respectively (FIGS. 5C, D).

### mCS NPs-KAg and CS NPs-KAg Vaccines Reduced/Cleared the Challenge SwIAV

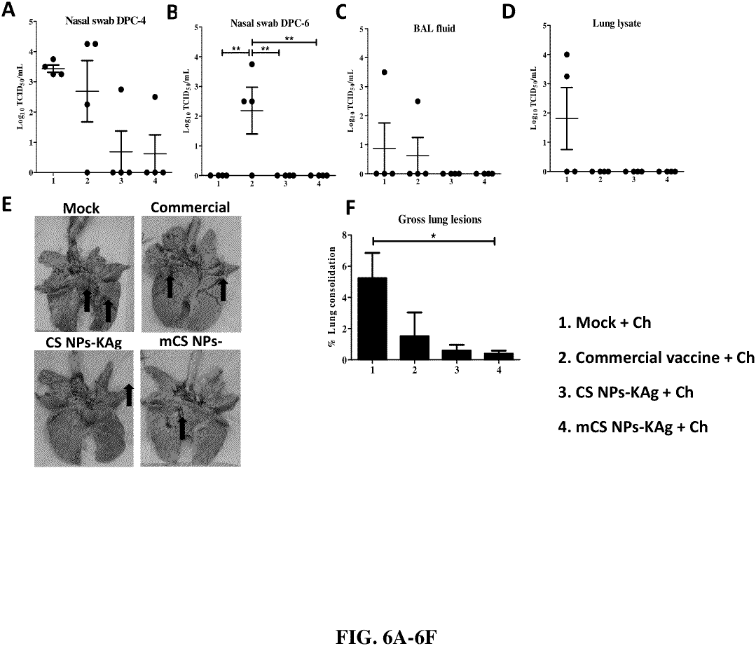

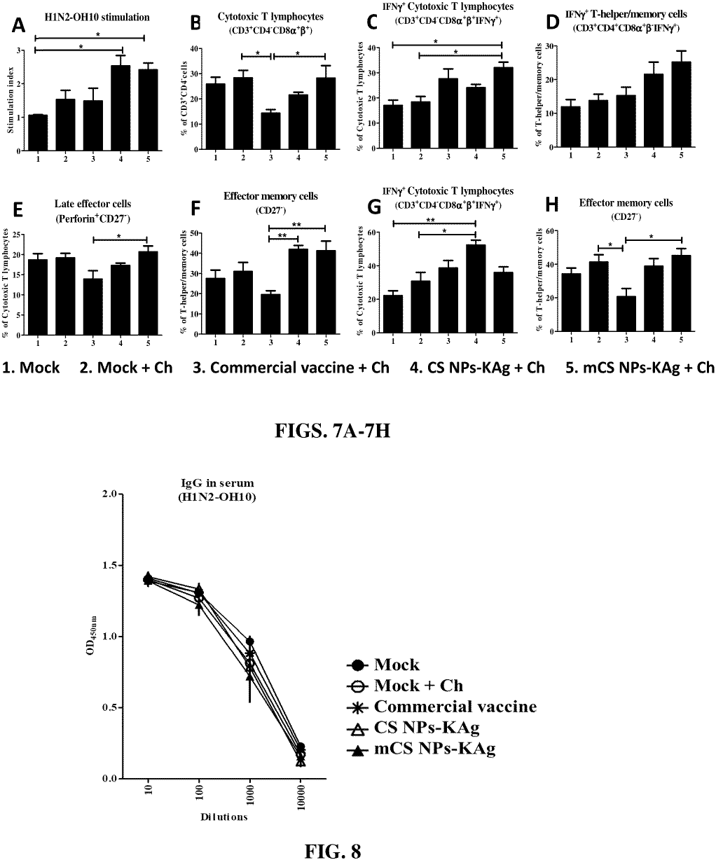

To evaluate whether the mCS NPs-KAg and CS NPs-KAg vaccines-induced cross-reactive antibodies in nasal passage and lungs were translated into the cross-protection, H1N1-OH7 challenge virus titers in the airways were examined. The virus load in nasal swab at DPC-4 was completely cleared in 3 of 4 pigs vaccinated with mCS NPs-KAg and CS NPs-KAg vaccines (FIG. 6A). While both mock-challenge and commercial vaccine received MDA-positive pigs had a higher virus load at DPC-4 in the nasal passage (FIG. 6A). Six days after challenge (DPC-6), infectious virus was absent in mock-challenge, mCS NPs-KAg and CS NPs-KAg vaccinates and the data was significant (p < 0.01) compared to commercial vaccinates (FIG. 6B). The challenge virus was undetectable in BAL fluid of mCS NPs-KAg and CS NPs-KAg vaccine inoculated animals, whereas one pig in both mock-challenge and commercial vaccine group had higher virus titer (FIG. 6C). In the mCS NPs-KAg, CS NPs-KAg, and commercial vaccines received pigs at DPC-6 the replicating virus in lung lysate was undetectable, while higher virus titer was noticed in two of the mock-challenge animals (FIG. 6D). Even though visible clinical signs were not observed in any of the virus challenged pig groups, consistent with the data of virus load in the airways, both the mCS NPs-KAg and CS NPs-KAg vaccinates had reduced macroscopic gross lung lesions (FIGS. 6E, F), with mCS NPs-KAg group data significantly (p < 0.05) lower compared to mock-challenge animals (FIG. 6F).

### Discussion

Inactivated influenza virus vaccine administration by the parenteral route is frequently ineffective due to the presence of maternally-derived antibodies (MDA) in weaned animals. As a result, effective protection is absent by vaccine-induced immune responses (Markowska-Daniel et al., 2011;Sandbulte et al., 2014;Zhang et al., 2016). Vaccination of pigs in the presence of MDA negatively impacts the vaccine efficacy, leads to lengthening of the clinical signs and development of SwIAV infection induced pneumonia (Kitikoon et al., 2006;Vincent et al., 2012). Mostly, MDA is the IgG class antibody primarily transferred from mothers to offspring through colostrum. Predominantly MDA influence the vaccine-induced IgG but not local sIgA antibody production in young piglets (Zhang et al., 2016). Further, mucosal vaccines elicit sIgA antibody responses that are independent by MDA-IgG levels in blood (Hill et al., 2012;Zhang et al., 2016).

The natural mucoadhesive properties of CS NPs have been widely used for vaccine delivery to mucosal surfaces (Renu et al., 2020a;Renu et al., 2020d). CS NPs-based vaccines administered IN enhance immunogenicity of entrapped antigens and elicit robust cross-reactive sIgA antibody response (Dhakal et al., 2018;Bagheripour et al., 2019). In mice, mannose ligand modified CS NPs administered mucosally specifically targets and delivers loaded antigen to the DCs (Chen et al., 2018). The present objectives were to evaluate the mCS NPs-KAg vaccine-induced immune responses and protection in MDA-positive pigs delivered intranasally (IN), and to compare the efficiency with CS NPs-KAg vaccine and parenterally administered multivalent commercial inactivated SwIAV vaccine.

Both mCS NPs-KAg and CS NPs-KAg vaccines delivered IN to MDA-positive pigs enhanced the cross-reactive sIgA in nasal passage, and sIgA and IgG antibodies in lung lysate and BAL fluid compared to the commercial vaccine. Particularly, mCS NPs-KAg vaccine compared to CS NPs-KAg vaccine induced increased trend (non-significant) in cross-reactive sIgA after prime-boost vaccination in nasal passage, and after virus challenge both the sIgA and IgG antibodies responses were significantly increased in BAL fluid. The IM-administered commercial vaccine increased cross-reactive IgG antibody responses and the sIgA antibody level was lower than mCS NPs-KAg vaccine. Consistent with previous studies in MDA-negative pigs (Renu et al., 2020b) the commercial SwIAV vaccine did not induce significantly higher mucosal antibody responses in MDA-positive animals. In another study, adjuvanted influenza whole-inactivated virus administered pigs by IM route in both MDA-positive and -negative pigs there was absence of sIgA antibody response (Sandbulte et al., 2014).

The protein antigen loaded mannosylated chitosan microspheres binds with MR on macrophages, and in mice IN vaccinated, it induces high levels of antigen specific sIgA antibody response (Jiang et al., 2008). Mannose modified CS NPs based vaccine promotes maturation and antigen presentation ability of DCs in vitro, and in vivo facilitates uptake of antigens by endogenous DCs within the draining lymph nodes in mice (Shi et al., 2017). Mannosylated nanoparticles internalized by macrophages induce its maturation through activation of MHC class I and II molecules in vitro (Zhu et al., 2020).

In recent studies (Renu et al., 2020a;Renu et al., 2020c), the CS NPs preparation method was optimized and achieved a monodispersed particle of uniform size and high positive surface charge. A similar method was adapted to prepare both the mCS NPs-KAg and CS NPs-KAg vaccines which induced both cross-reactive sIgA and IgG antibodies in the lungs. In an earlier study (Dhakal et al., 2018), polydispersed and particles carrying relatively less positive surface charge on CS NPs-KAg vaccine induced a lesser cross-reactive IgG antibody response in IN vaccinated pigs, suggesting that size and charge of CS NPs-KAg vaccine are important to elicit a broader immune response. Further, the optimized CS NPs-KAg vaccine co-delivered with a Th1 response promoting adjuvant poly(I:C) did not induce higher cross-reactive mucosal immune responses in pigs (Renu et al., 2020a), suggesting the proper selection of secondary adjuvant is critical to achieve robust mucosal immune responses. Both in mice and chickens, CS NPs based influenza vaccine administered IN increased systemic IgG and mucosal sIgA antibodies (Sawaengsak et al., 2014;Hajam et al., 2020). This example in MDA-positive young swine confirmed the potent immunogenicity of mCS NPs-KAg and CS NPs-KAg vaccines when delivered IN resulting in increased breadth of mucosal and systemic immune responses.

In the present example, both the mCS NPs-KAg and CS NPs-KAg vaccines in MDA-positive pigs increased the vaccine (H1N2-OH10) virus-specific serum HI titers, results similar to influenza free specific pathogen-free swine given CS NPs-KAg vaccine (Dhakal et al., 2018). Likewise, CS NPs-KAg and poly(I:C) co-administration IN in pigs increased the H1N2-OH10 virus specific serum HI titer (Renu et al., 2020a).

In lung draining TBLN, both mCS NPs-KAg and CS NPs-KAg vaccines increased the recall cell proliferation, and especially the former augmented the cytokine (IL-4, IL-10, and IFNγ) gene expression. Consistent with the present study, in another vaccine trial (Renu et al., 2020b) the commercial SwIAV vaccine did not augment Th1 and Th2 cytokines gene expression. Studies have shown that influenza virus-specific effector T cells help in the clearance of virus by triggering the expression of cytokines IFNy, TNFα, IL-4 and IL-10 (Russell and Ley, 2002;Teijaro et al., 2010;Spitaels et al., 2016). As well, upon reinfection memory cells produced the effector T cells to facilitate control of the infection (Welsh and McNally, 1999).

Chitosan enhances T cell responses by promoting the maturation of DCs by signaling nascent DCs in a type I IFN receptor-dependent manner (Carroll et al., 2016). Co-immunization of CS NPs-KAg and poly (I:C) vaccine enhanced the Th1 and Th2 cytokines gene expression in pigs (Renu et al., 2020a). Mannan conjugated antigen hasten the MHC class I presentation to CD8⁺ T cells, leads to Th1 immune response (Apostolopoulos et al., 2000). The Th2 cytokines IL-4 and L-10 are the driving factor to upregulate MR expression in macrophages (Martinez-Pomares et al., 2003), and higher MR expression is associated with the induction of Th2 mediated immune response (van Die and Cummings, 2017).

The mCS NPs-KAg and CS NPs-KAg vaccines enhanced cross-reactive sIgA and IgG antibody levels in both the nasal passage and lungs (lung lysate and BAL fluid), which, together with increased cellular responses, resulted in reduced/cleared challenge heterologous (H1N1-OH7) SwIAV from both the upper and lower respiratory tract. Furthermore, mCS NPs-KAg and CS NPs-KAg vaccines reduced the influenza virus titers correlated with decreased macroscopic lung lesions. Earlier studies have established that circulating sIgA and IgG antibodies induced by an IN vaccination has been correlated with protection against influenza virus infection in mice, chicken, pigs, and humans (Belshe et al., 2000;Sawaengsak et al., 2014;Dhakal et al., 2018;Hajam et al., 2020). Antigen specific sIgA antibody more efficiently prevents the influenza virus infection of mucosal surfaces than does circulating systemic IgG antibody (Muramatsu et al., 2014;Gould et al., 2017). The innate and cell-mediated immunity also plays a key role in the clearance of influenza viral infection from infected tissues (Forrest et al., 2008;Bahadoran et al., 2016).

Consistent with an earlier study (Kitikoon et al., 2006), commercial inactivated influenza virus vaccine in the present example did not provide a protection of upper respiratory tract infection in pigs. An IM immunization of commercial inactivated influenza virus vaccine in MDA-positive pigs boosts HI titer, but not sIgA and indices of the cellular immune responses and thus failed to provide protection against heterologous virus infections (Kitikoon et al., 2006). A study has shown that adjuvanted whole inactivated influenza virus vaccine administered IM in MDA-positive pigs dramatically increased a phenomenon known as vaccine-associated enhanced respiratory disease following heterologous virus challenge (Vincent et al., 2012).

### Conclusions

The mannose conjugated CS NPs delivered monovalent inactivated SwIAV vaccine administered IN in MDA-positive pigs augmented the homologous, heterologous, and heterosubtypic virus specific mucosal sIgA and IgG and systemic IgG antibodies in airways. The mCS NPs-KAg vaccine increased specific recall cell proliferation and cytokine gene expression in the tracheobronchial lymph nodes resulting in reduced/cleared heterologous challenge virus infection. Overall, this example shows that mCS NPs-KAg vaccine IN delivery is a useful and effective alternative to commercial influenza vaccines for inducing cross-protective immunity against SwIAVs in MDA-positive grower finisher pigs.

### References Cited in This Example

Akerele, G., Ramadan, N., Renu, S., Renukaradhya, G.J., Shanmugasundaram, R., and Selvaraj, R.K. (2020). In vitro characterization and immunogenicity of chitosan nanoparticles loaded with native and inactivated extracellular proteins from a field strain of Clostridium perfringens associated with necrotic enteritis. Veterinary Immunology and Immunopathology 224, 110059.

Apostolopoulos, V., Pietersz, G.A., Gordon, S., Martinez-Pomares, L., and Mckenzie, I.F.C. (2000). Aldehyde-mannan antigen complexes target the MHC class I antigen-presentation pathway. European Journal of Immunology 30, 1714-1723.

Apostolopoulos, V., Thalhammer, T., Tzakos, A.G., and Stojanovska, L. (2013). Targeting antigens to dendritic cell receptors for vaccine development. Journal of drug delivery 2013, 869718-869718.

Bagheripour, M.J., Ebrahimi, F., Hajizade, A., and Nazarian, S. (2019). Immunogenicity evaluation of rBoNT/E nanovaccine after mucosal administration. Iranian journal of basic medical sciences 22, 353-359.

Bahadoran, A., Lee, S.H., Wang, S.M., Manikam, R., Rajarajeswaran, J., Raju, C.S., and Sekaran, S.D. (2016). Immune Responses to Influenza Virus and Its Correlation to Age and Inherited Factors. Frontiers in microbiology 7, 1841-1841.

Belshe, R.B., Gruber, W.C., Mendelman, P.M., Mehta, H.B., Mahmood, K., Reisinger, K., Treanor, J., Zangwill, K., Hayden, F.G., Bernstein, D.I., Kotloff, K., King, J., Piedra, P.A., Block, S.L., Yan, L., and Wolff, M. (2000). Correlates of Immune Protection Induced by Live, Attenuated, Cold-Adapted, Trivalent, Intranasal Influenza Virus Vaccine. The Journal of Infectious Diseases 181, 1133-1137.

Carroll, E.C., Jin, L., Mori, A., Munoz-Wolf, N., Oleszycka, E., Moran, H.B.T., Mansouri, S., Mcentee, C.P., Lambe, E., Agger, E.M., Andersen, P., Cunningham, C., Hertzog, P., Fitzgerald, K.A., Bowie, A.G., and Lavelle, E.C. (2016). The Vaccine Adjuvant Chitosan Promotes Cellular Immunity via DNA Sensor cGAS-STING-Dependent Induction of Type I Interferons. Immunity 44, 597-608.

Chamba Pardo, F.O., Wayne, S., Culhane, M.R., Perez, A., Allerson, M., and Torremorell, M. (2019). Effect of strain-specific maternally-derived antibodies on influenza A virus infection dynamics in nursery pigs. PLOS ONE 14, e0210700.

Chaubey, P., and Mishra, B. (2014). Mannose-conjugated chitosan nanoparticles loaded with rifampicin for the treatment of visceral leishmaniasis. Carbohydr Polym 101, 1101-1108.

Chen, Y., Wu, J., Wang, J., Zhang, W., Xu, B., Xu, X., and Zong, L. (2018). Targeted delivery of antigen to intestinal dendritic cells induces oral tolerance and prevents autoimmune diabetes in NOD mice. Diabetologia 61, 1384-1396.

Cox, R.J., Brokstad, K.A., and Ogra, P. (2004). Influenza Virus: Immunity and Vaccination Strategies. Comparison of the Immune Response to Inactivated and Live, Attenuated Influenza Vaccines. Scandinavian Journal of Immunology 59, 1-15.

Cui, Z., Hsu, C.-H., and Mumper, R.J. (2003). Physical Characterization and Macrophage Cell Uptake of Mannan-Coated Nanoparticles. Drug Development and Industrial Pharmacy 29, 689-700.

Deblanc, C., Hervé, S., Gorin, S., Cador, C., Andraud, M., Quéguiner, S., Barbier, N., Paboeuf, F., Rose, N., and Simon, G. (2018). Maternally-derived antibodies do not inhibit swine influenza virus replication in piglets but decrease excreted virus infectivity and impair post-infectious immune responses. Veterinary Microbiology 216, 142-152.

Dhakal, S., Hiremath, J., Bondra, K., Lakshmanappa, Y.S., Shyu, D.L., Ouyang, K., Kang, K.I., Binjawadagi, B., Goodman, J., Tabynov, K., Krakowka, S., Narasimhan, B., Lee, C.W., and Renukaradhya, G.J. (2017). Biodegradable nanoparticle delivery of inactivated swine influenza virus vaccine provides heterologous cell-mediated immune response in pigs. J Control Release 247, 194-205.

Dhakal, S., Renu, S., Ghimire, S., Shaan Lakshmanappa, Y., Hogshead, B.T., Feliciano-Ruiz, N., Lu, F., Hogenesch, H., Krakowka, S., Lee, C.W., and Renukaradhya, G.J. (2018). Mucosal Immunity and Protective Efficacy of Intranasal Inactivated Influenza Vaccine Is Improved by Chitosan Nanoparticle Delivery in Pigs. Front Immunol 9, 934.

Engering, A.J., Cella, M., Fluitsma, D., Brockhaus, M., Hoefsmit, E.C.M., Lanzavecchia, A., and Pieters, J. (1997). The mannose receptor functions as a high capacity and broad specificity antigen receptor in human dendritic cells. European Journal of Immunology 27, 2417-2425.

Forrest, B.D., Pride, M.W., Dunning, A.J., Capeding, M.R.Z., Chotpitayasunondh, T., Tam, J.S., Rappaport, R., Eldridge, J.H., and Gruber, W.C. (2008). Correlation of Cellular Immune Responses with Protection against Culture-Confirmed Influenza Virus in Young Children. Clinical and Vaccine Immunology 15, 1042-1053.

Gao, F., Xie, J.L., Jia, C.W., Ren, H.Y., and Zhou, S.H. (2014). Effects of porcine circovirus type 2 and pseudorabies vaccine co-inoculation on regulatory cytokine mRNA expression in pig peripheral blood mononuclear cells. Genet Mol Res 13, 1540-1547.

Gould, V.M.W., Francis, J.N., Anderson, K.J., Georges, B., Cope, A.V., and Tregoning, J.S. (2017). Nasal IgA Provides Protection against Human Influenza Challenge in Volunteers with Low Serum Influenza Antibody Titre. Frontiers in Microbiology 8.

Gray, G.C., Mccarthy, T., Capuano, A.W., Setterquist, S.F., Olsen, C.W., and Alavanja, M.C. (2007). Swine workers and swine influenza virus infections. Emerging infectious diseases 13, 1871-1878.

Hajam, I.A., Senevirathne, A., Hewawaduge, C., Kim, J., and Lee, J.H. (2020). Intranasally administered protein coated chitosan nanoparticles encapsulating influenza H9N2 HA2 and M2e mRNA molecules elicit protective immunity against avian influenza viruses in chickens. Vet Res 51, 37.

Han, Y., Renu, S., Patil, V., Schrock, J., Feliciano-Ruiz, N., Selvaraj, R., and Renukaradhya, G.J. (2020). Immune Response to Salmonella Enteritidis Infection in Broilers Immunized Orally With Chitosan-Based Salmonella Subunit Nanoparticle Vaccine. Frontiers in Immunology 11.

Hill, K.L., Hunsaker, B.D., Townsend, H.G., Van Drunen Littel-Van Den Hurk, S., and Griebel, P.J. (2012). Mucosal immune response in newborn Holstein calves that had maternally derived antibodies and were vaccinated with an intranasal multivalent modified-live virus vaccine. Journal of the American Veterinary Medical Association 240, 1231-1240.

Hiremath, J., Kang, K.I., Xia, M., Elaish, M., Binjawadagi, B., Ouyang, K., Dhakal, S., Arcos, J., Torrelles, J.B., Jiang, X., Lee, C.W., and Renukaradhya, G.J. (2016). Entrapment of H1N1 Influenza Virus Derived Conserved Peptides in PLGA Nanoparticles Enhances T Cell Response and Vaccine Efficacy in Pigs. PLoS One 11, e0151922.

Jiang, H.-L., Kang, M.L., Quan, J.-S., Kang, S.G., Akaike, T., Yoo, H.S., and Cho, C.-S. (2008). The potential of mannosylated chitosan microspheres to target macrophage mannose receptors in an adjuvant-delivery system for intranasal immunization. Biomaterials 29, 1931-1939.

Karasin, A.I., Schutten, M.M., Cooper, L.A., Smith, C.B., Subbarao, K., Anderson, G.A., Carman, S., and Olsen, C.W. (2000). Genetic characterization of H3N2 influenza viruses isolated from pigs in North America, 1977-1999: evidence for wholly human and reassortant virus genotypes. Virus Research 68, 71-85.

Kitikoon, P., Nilubol, D., Erickson, B.J., Janke, B.H., Hoover, T.C., Sornsen, S.A., and Thacker, E.L. (2006). The immune response and maternal antibody interference to a heterologous H1N1 swine influenza virus infection following vaccination. Veterinary Immunology and Immunopathology 112, 117-128.

Li, M., Wang, Y., Sun, Y., Cui, H., Zhu, S.J., and Qiu, H.-J. (2020). Mucosal vaccines: Strategies and challenges. Immunology Letters 217, 116-125.

M Ways, T.M., Lau, W.M., and Khutoryanskiy, V.V. (2018). Chitosan and Its Derivatives for Application in Mucoadhesive Drug Delivery Systems. Polymers 10, 267.

Markowska-Daniel, I., Pomorska-Mól, M., and Pejsak, Z. (2011). The influence of age and maternal antibodies on the postvaccinal response against swine influenza viruses in pigs. Veterinary Immunology and Immunopathology 142, 81-86.

Martinez-Pomares, L., Reid, D.M., Brown, G.D., Taylor, P.R., Stillion, R.J., Linehan, S.A., Zamze, S., Gordon, S., and Wong, S.Y.C. (2003). Analysis of mannose receptor regulation by IL-4, IL-10, and proteolytic processing using novel monoclonal antibodies. Journal of Leukocyte Biology 73, 604-613.

Mastin, A., Alarcon, P., Pfeiffer, D., Wood, J., Williamson, S., Brown, I., Consortium, C., and Wieland, B. (2011). Prevalence and risk factors for swine influenza virus infection in the English pig population. PLoS Curr 3, RRN1209.

Muramatsu, M., Yoshida, R., Yokoyama, A., Miyamoto, H., Kajihara, M., Maruyama, J., Nao, N., Manzoor, R., and Takada, A. (2014). Comparison of antiviral activity between IgA and IgG specific to influenza virus hemagglutinin: increased potential of IgA for heterosubtypic immunity. PLoS One 9, e85582.

Newman, A.P., Reisdorf, E., Beinemann, J., Uyeki, T.M., Balish, A., Shu, B., Lindstrom, S., Achenbach, J., Smith, C., and Davis, J.P. (2008). Human case of swine influenza A (H1N1) triple reassortant virus infection, Wisconsin. Emerging infectious diseases 14, 1470-1472.

Peng, Y., Yao, W., Wang, B., and Zong, L. (2015). Mannosylated Chitosan Nanoparticles Based Macrophage-Targeting Gene Delivery System Enhanced Cellular Uptake and Improved Transfection Efficiency. Journal of Nanoscience and Nanotechnology 15, 2619-2627.

Pomorska-Mól, M., Dors, A., Kwit, K., Czyzewska-Dors, E., and Pejsak, Z. (2017). Coinfection modulates inflammatory responses, clinical outcome and pathogen load of H1N1 swine influenza virus and Haemophilus parasuis infections in pigs. BMC veterinary research 13, 376-376.

Renu, S., Feliciano-Ruiz, N., Ghimire, S., Han, Y., Schrock, J., Dhakal, S., Patil, V., Krakowka, S., and Renukaradhya, G.J. (2020a). Poly(I:C) augments inactivated influenza virus-chitosan nanovaccine induced cell mediated immune response in pigs vaccinated intranasally. Vet Microbiol 242, 108611.

Renu, S., Feliciano-Ruiz, N., Lu, F., Ghimire, S., Han, Y., Schrock, J., Dhakal, S., Patil, V., Krakowka, S., Hogenesch, H., and Renukaradhya, G.J. (2020b). A Nanoparticle-Poly(I:C) Combination Adjuvant Enhances the Breadth of the Immune Response to Inactivated Influenza Virus Vaccine in Pigs. Vaccines 8, 229.

Renu, S., Han, Y., Dhakal, S., Lakshmanappa, Y.S., Ghimire, S., Feliciano-Ruiz, N., Senapati, S., Narasimhan, B., Selvaraj, R., and Renukaradhya, G.J. (2020c). Chitosan-adjuvanted Salmonella subunit nanoparticle vaccine for poultry delivered through drinking water and feed. Carbohydrate Polymers, 116434.

Renu, S., Markazi, A.D., Dhakal, S., Lakshmanappa, Y.S., Gourapura, S.R., Shanmugasundaram, R., Senapati, S., Narasimhan, B., Selvaraj, R.K., and Renukaradhya, G.J. (2018). Surface engineered polyanhydride-based oral Salmonella subunit nanovaccine for poultry. Int J Nanomedicine 13, 8195-8215.

Renu, S., Markazi, A.D., Dhakal, S., Lakshmanappa, Y.S., Shanmugasundaram, R., Selvaraj, R.K., and Renukaradhya, G.J. (2020d). Oral Deliverable Mucoadhesive Chitosan-Salmonella Subunit Nanovaccine for Layer Chickens. Int J Nanomedicine 15, 761-777.

Russell, J.H., and Ley, T.J. (2002). Lymphocyte-mediated cytotoxicity. Annu Rev Immunol 20, 323-370.

Sandbulte, M.R., Platt, R., Roth, J.A., Henningson, J.N., Gibson, K.A., Rajão, D.S., Loving, C.L., and Vincent, A.L. (2014). Divergent immune responses and disease outcomes in piglets immunized with inactivated and attenuated H3N2 swine influenza vaccines in the presence of maternally-derived antibodies. Virology 464-465, 45-54.

Sawaengsak, C., Mori, Y., Yamanishi, K., Mitrevej, A., and Sinchaipanid, N. (2014). Chitosan nanoparticle encapsulated hemagglutinin-split influenza virus mucosal vaccine. AAPS PharmSciTech 15, 317-325.

Schultz-Cherry, S., Olsen, C.W., and Easterday, B.C. (2013). “History of Swine Influenza,” in Swine Influenza, eds. J.A. Richt & R.J. Webby. (Berlin, Heidelberg: Springer Berlin Heidelberg), 21-27.

Shi, G.-N., Zhang, C.-N., Xu, R., Niu, J.-F., Song, H.-J., Zhang, X.-Y., Wang, W.-W., Wang, Y.-M., Li, C., Wei, X.-Q., and Kong, D.-L. (2017). Enhanced antitumor immunity by targeting dendritic cells with tumor cell lysate-loaded chitosan nanoparticles vaccine. Biomaterials 113, 191-202.

Spitaels, J., Roose, K., and Saelens, X. (2016). Influenza and Memory T Cells: How to Awake the Force. Vaccines 4, 33.

Sunwoo, S.-Y., Schotsaert, M., Morozov, I., Davis, A.S., Li, Y., Lee, J., Mcdowell, C., Meade, P., Nachbagauer, R., García-Sastre, A., Ma, W., Krammer, F., and Richt, J.A. (2018). A Universal Influenza Virus Vaccine Candidate Tested in a Pig Vaccination-Infection Model in the Presence of Maternal Antibodies. Vaccines 6, 64.

Teijaro, J.R., Verhoeven, D., Page, C.A., Turner, D., and Farber, D.L. (2010). Memory CD4 T Cells Direct Protective Responses to Influenza Virus in the Lungs through Helper-Independent Mechanisms. Journal of Virology 84, 9217-9226.

Thomas, F.P., Terry, L.N., and Jennifer, L.R. (2010). One Health Approach to Influenza: Assessment of Critical Issues and Options. Emerging Infectious Disease journal 16.

Tokatlian, T., Read, B.J., Jones, C.A., Kulp, D.W., Menis, S., Chang, J.Y.H., Steichen, J.M., Kumari, S., Allen, J.D., Dane, E.L., Liguori, A., Sangesland, M., Lingwood, D., Crispin, M., Schief, W.R., and Irvine, D.J. (2019). Innate immune recognition of glycans targets HIV nanoparticle immunogens to germinal centers. Science 363, 649-654.

Trebbien, R., Larsen, L.E., and Viuff, B.M. (2011). Distribution of sialic acid receptors and influenza A virus of avian and swine origin in experimentally infected pigs. Virol J 8, 434.

Van Die, I., and Cummings, R.D. (2017). The Mannose Receptor in Regulation of Helminth-Mediated Host Immunity. Frontiers in immunology 8, 1677-1677.

Vincent, A.L., Ma, W., Lager, K.M., Janke, B.H., and Richt, J.A. (2008). Swine influenza viruses a North American perspective. Adv Virus Res 72, 127-154.

Vincent, A.L., Ma, W., Lager, K.M., Richt, J.A., Janke, B.H., Sandbulte, M.R., Gauger, P.C., Loving, C.L., Webby, R.J., and García-Sastre, A. (2012). Live attenuated influenza vaccine provides superior protection from heterologous infection in pigs with maternal antibodies without inducing vaccine-associated enhanced respiratory disease. Journal of virology 86, 10597-10605.

Welsh, R.M., and Mcnally, J.M. (1999). Immune deficiency, immune silencing, and clonal exhaustion of T cell responses during viral infections. Curr Opin Microbiol 2, 382-387.

Zaman, M., Chandrudu, S., and Toth, I. (2013). Strategies for intranasal delivery of vaccines. Drug delivery and translational research 3, 100-109.

Zhang, F., Peng, B., Chang, H., Zhang, R., Lu, F., Wang, F., Fang, F., and Chen, Z. (2016). Intranasal Immunization of Mice to Avoid Interference of Maternal Antibody against H5N1 Infection. PloS one 11, e0157041-e0157041.

Zhu, B., Zhang, C., Zhao, Z., and Wang, G.-X. (2020). Targeted Delivery of Mannosylated Nanoparticles Improve Prophylactic Efficacy of Immersion Vaccine against Fish Viral Disease. Vaccines 8, 87.

Publications cited herein are hereby specifically incorporated by reference in their entireties and at least for the material for which they are cited.

It should be understood that while the present disclosure has been provided in detail with respect to certain illustrative and specific aspects thereof, it should not be considered limited to such, as numerous modifications are possible without departing from the broad spirit and scope of the present disclosure as defined in the appended claims. It is, therefore, intended that the appended claims cover all such equivalent variations as fall within the true spirit and scope of the invention.

What is claimed is:

1. A nanoparticle comprising mannose conjugated chitosan and an inactivated influenza A virus (IAV) antigen, wherein the mannose conjugated chitosan encapsulates the inactivated IAV antigen.

2. The nanoparticle of claim 1, further comprising tripolyphosphate.

3. The nanoparticle of claim 1, wherein the nanoparticle has a diameter of 500 nm or less.

4. The nanoparticle of claim 1, wherein the nanoparticle has an encapsulation efficiency of inactivated IAV antigen of at least 60%.

5. The nanoparticle of claim 1, wherein the inactivated IAV antigen comprises an inactivated swine IAV antigen.

6. The nanoparticle of claim 1, wherein the inactivated IAV antigen is from an H1N1, H1N2 or H3N2 virus.

7. The nanoparticle of claim 1, wherein the inactivated IAV antigen is homologous, heterologous, or heterosubtypic to an influenza A virus.

8. The nanoparticle of claim 7, wherein the nanoparticle reduces transmission of the influenza A virus.

9. The nanoparticle of claim 7, wherein the nanoparticle reduces nasal shedding of the influenza A virus compared to a control.

10. The nanoparticle of claim 9, wherein the nanoparticle reduces the amount of the influenza A virus in an upper respiratory tract of a subject by at least 1x10¹ TCID₅₀/mL compared to a control.

11. The nanoparticle of claim 9, wherein the reduction in the amount of the influenza A virus occurs within four days of exposure to the influenza A virus.

12. The nanoparticle of claim 1, wherein the nanoparticle elicits an increased amount of IgA antibody in a subject compared to a control.

13. The nanoparticle of claim 12, wherein the increased amount of IgA antibody occurs in mucosa.

14. A vaccine comprising a composition of claim 1, in a pharmaceutically acceptable carrier.

15. A method of reducing transmission of an influenza A virus in a subject compared to a control comprising administering to the subject a nanoparticle comprising mannose conjugated chitosan and an inactivated influenza A virus (IAV) antigen, wherein the mannose conjugated chitosan encapsulates the inactivated IAV antigen.

16. The method of claim 15, wherein the nanoparticle is administered intranasally.

17. The method of claim 15, wherein the subject is a pig.

18. A method of eliciting an immune response against swine influenza A virus in a subject comprising administering to the subject a nanoparticle comprising mannose conjugated chitosan and an inactivated influenza A virus (IAV) antigen, wherein the mannose conjugated chitosan encapsulates the inactivated IAV antigen.

19. The method of claim 18, wherein the method elicits an increased amount of IgA antibody in the subject compared to a control.

20. The method of claim 18, wherein the increased amount of IgA antibody occurs in a respiratory system location comprising upper respiratory tract, lower respiratory tract, or lung parenchyma.
